(19) United States
(12) Patent Application Publication
(10) Pub. No.: US 2021/0137948 A1
(43) Pub. Date: May 13, 2021

(54) TREATMENT OF INFECTIONS CAUSED BY NEISSERIA GONOCOCCUS USING A HALOGENATED SALICYLANILIDE

(71) Applicant: UNION therapeutics A/S, Hellerup (DK)

(72) Inventor: Morten Otto Alexander SOMMER, Hellerup (DK)

(21) Appl. No.: 17/044,377

(22) PCT Filed: Apr. 1, 2019
(86) PCT No.: PCT/EP2019/058202

§ 371 Date: Oct. 1, 2020

(30) Foreign Application Priority Data
Apr. 3, 2018 (GB) 1805453.6

(51) Int. Cl. A61K 31/609; A61K 9/00; A61K 45/06

(52) U.S. Cl. CPC A61K 31/609; A61K 45/06; A61K 9/0048; A61K 9/0014

(57) ABSTRACT

The present invention relates to halogenated salicylanilides, such as closantel, rafoxanide, oxyclozanide, niclosamide, or pharmaceutically acceptable salts or solvates thereof, for use in the treatment of an infection caused by N. gonococcus bacteria in a subject, such as treatment of gonococcal conjunctivitis or anorectal gonorrhoea.

This disclosure relates to halogenated salicylanilides, in particular closantel, rafoxanide, oxyclozanide, niclosamide, or pharmaceutically acceptable salts or solvates thereof, for use in the treatment, including prevention, of an infection caused by Neisseria gonococcus (also known as Neisseria gonorrhoeae or Neisseria gonorrhoea).

### BACKGROUND

Gonorrhea is one of the most commonly reported communicable diseases. In July 2017, the World Health Organization (WHO) reported that about 78 million people are infected with gonorrhea each year, and that 97% of 77 countries surveyed from 2009 to 2014 reported the presence of drug-resistant gonorrhea strains.

Gonorrhea (gonorrhoea) is a sexually treansmitted infection (STI) caused by the bacterium Neisseria gonococcus, herein also referred to as N. gonococcus. N. gonococcus can also be transmissioned from mother to child during childbirth.

Bacteria of the genus Neisseria are Gram-negative proteobacteria and include about 11 species that colonize humans, but Neisseria gonococcus and Neisseria meningitidis are considered the primary pathogens.

N. gonococcus infections are often classified as either uncomplicated or complicated. Uncomplicated infections are infections caused by N. gonococcus that does not result in bacteremia. Bacteremia is the presence of bacteria in the blood. An N. gonococcus infection that results in bacteremia and/or the spread of bacteria to joints and tissues is considered a complicated infection.

N. gonococcus infections may cause uncomplicated urogenital (including endocervical/cervical), anorectal, conjunctival, and/or pharyngeal infections. Thus, the infection may affect multiple organs including urethra, ovarian/uterine, cervix, epididymis, prostate glands, rectum, throat/pharynx and/or eyes and cause morbidity in humans. Urogenital tract infections are most common. Often, N. gonococcus infection causes symptoms, but infections can be asymptomatic. Extra-genital infections are common in both men and women and often occur in the absence of urogenital infection. Anorectal and pharyngeal infections are mostly asymptomatic but mild symptoms may occur. In the absence of symptoms, anorectal and pharyngeal infections often remain untreated, despite their key role in disease transmission.

Untreated or undertreated N. gonococcus infections may lead to a complicated gonococcal infection; a so-called disseminated gonococcal infection (DGI). DGI is rare and affects only less than 1% of gonorrhoea patients. DGI, also called systemic gonococcal infection, occurs when gonorrhea bacteria enter the bloodstream. Complications may include gonococcal arthritis, and, although rare, gonococcal endocarditis or gonococcal meningitis. The most common complication of disseminated gonococcal infection is arthritis-dermatitis syndrome, including joint or tendon pain and eventually septic arthritis. The knee is commonly affected by gonococcal arthritis.

Untreated gonorrhea may also increase the risk for the transmission and acquisition of HIV, which is the virus that causes AIDS.

Coinfection with Herpes Simplex Virus, Chlamydia trachomatis (also referred to as C. trachomatis), Mycoplasma genitalium (also referred to as M. genitalium), and syphilis are common in individuals infected with N. gonococcus and may result in synergistic effects on transmission and disease severity.

Uncomplicated gonorrhoea affecting the lower genital tract is a reasonably common infection but complicated gonorrhoea affecting the upper genital tract, such as pelvic inflammatory disease (PID), is rarer.

Among women, urogenital gonococcal infections are commonly asymptomatic or might not produce recognizable symptoms. Genitourinary symptoms of gonorrhea in women include abnormal vaginal discharge, dysuria, intermenstrual bleeding, dyspareunia (painful intercourse) and mild lower abdominal pain. Untreated or under-treated infections can in women lead to, for instance, pelvic inflammatory disease (PID), which can result in, for instance, chronic pelvic pain, and sterility.

Urogenital infections caused by N. gonococcus among men generellay produce symptoms that cause them to seek curative treatment. Genitourinary symptoms of gonorrhoea in men include penile discharge, dysuria urethritis, epididymitis, reduced fertility, urethral strictures, and rectal infection.

In conjunctivitis infection by N. gonococcus results in inflammation of conjunctiva of the eye. Often the white areas of the eye turn red or pink, and it is therefore also known as “pink eye”. N. gonococcus may infect the eye and and cause gonococcal conjunctivitis (also called gonorrheal conjunctivitis). The infection may affect either one or both the eyes and is accompanied by inflammation, irritation, eye pain, and discharge from the eye. Gonorrhea can also be transmitted from mother's genital tract to the newborn baby during birth, causing neonatal gonococcal conjunctivitis (also called opthalmia neonatorum) and/or systemic neonatal gonococcal infection. In neonates, neonatal gonococcal conjunctivitis can lead to blindness. The infection may also cause scalp sores with babies.

Most anorectal N. gonococcus infections are asymptomatic, but symptoms can include anal pruritus, rectal pain, mucopurulent discharge, and tenesmus. Untreated rectal infections cause symptomatic proctitis.

Gonorrhea can be transmitted through unprotected sexual contact with the oropharynx. Most pharyngeal gonorrhea is asymptomatic. Pharyngeal gonorrhea is more challenging to treat than urogenital infections.

Antibiotic treatment is the only option to alleviate symptoms and cure the N. gonococcus infection, thereby reducing the risk of complications, and end further transmission of the infection. However, gonorrhea treatment is complicated by the ability of N. gonococcus to develop resistance to antibiotics.

During the 1930s, sulfonamides were the first therapy introduced as a therapy for gonorrhea. However, there was a rapid emergence of resistance against sulfonamides. A synergistic combination of sulfonamides with trimethoprim was used to treat uncomplicated gonorrhea until the 1970s.

During the 1940s, penicillin replaced sulfonamide for treatment of gonorrhoea. However, N. gonococcus gradually developed also resistance against penicillin.

During the 1950s, tetracycline (e.g. doxycycline) emerged as an alternative antibiotic for gonorrhea. However, tetracycline resistance also soon developed.

During the 1980s, oral fluoroquinolones (e.g. ciprofloxacin, levofloxacin, and ofloxacin) was introduced. By early to mid 2000s, recommendation to use fluoroquinolone was withdrawn due to development of resistant Neisseria gonococcus in patients.

N. gonococcus has rapidly developed resistance to many currently approved antimicrobial treatments. For example, N. gonococcus resistance to sulphonamides, penicillins, earlier generation cephalosporins, tetracyclines, macrolides and fluoroquinolones, is high all over the world. In most countries, only extended-spectrum cephalosporins, such as cefixime (oral) and ceftriaxone (injectable intramuscularaly or intravenously), is recommended for N. gonococcus treatment. However, it has been observed that the effectiveness of cefixime might also be waning.

A combination therapy using two antibiotics with different mechanisms of action is often used to improve treatment efficacy and potentially slow the emergence and spread of resistance. The macrolide azithromycin was included as a possible therapy for gonorrhea in the beginning of the 1980s. Treatment guidelines have therefore recommended dual therapy for gonorrhea with a cephalosporin (cefixime or ceftriaxone) plus either azithromycin or doxycycline. However, resistance is also emerging towards such combination treatments (Fifer et al, N. Engl. J. Med. 374:25, p. 2504-2506).

Azithromycin is preferred over doxycycline because of the high prevalence of tetracycline resistance. Persons infected with N. gonococcus frequently are coinfected with C. trachomatis, which has led to the longstanding recommendation that persons treated for uncomplicated cervical, urethral, anorectal, and pharyngeal gonococcal infection also should be treated with a regimen that is effective against uncomplicated genital C. trachomatis infection, further supporting the use of dual therapy that includes ceftriaxone and azithromycin. Cases of resistance to ceftriaxone have been reported but are still rare.

Thus, gonorrhea has the potential to become untreatable with existing medicaments (M. Unemo, BMC Infect. Dis., 15:364, 2015). Given the significant risk that antibiotic resistance presents to human and animal health, there is a need for new approaches to treat and prevent infections caused by Neisseria gonococcus.

Halogenated salicylanilides such as oxyclozanide, niclosamide, closantel and rafoxanide, are important anthelmintics that are used extensively in the control of Haemonchus spp. and Fasciola spp. infestation in sheep and cattle, and Oestrus ovis in sheep.

Niclosamide is commercially available in a number of formulations including, but not limited to Bayer73®, Bayer2353®, Bayer25648®, Bayluscid®, Baylucide®, Cestocid®, Clonitralid, Dichlosale®, Fenasal®, HL 2447®, lomesan®, lomezan®, Manosil®, Nasemo®, Niclosamid®, Phenasal®, Tredemine®, Sulqui®, Vermitid®, Vermitin® and Yomesan®.

Niclosamide has been proposed as a possible systemic treatment for chronic lung infections caused by the proteobacterium Pseudomonas aeruginosa and the actinobacterium Mycoplasmum tuberculosis (F. Imperi et al., Antimicrobial, Agents and Chemotherapy, 557(2), 996-1005 (2013)).

J. Vinsova et al. (Molecules, vol. 12, no. 1, pp. 1-12, 2007; Bioorganic and Medicinal Chemistry Letters, vol. 19, no. 2, pp. 348-351, 2009; European Journal of Medicinal Chemistry, vol. 45, no. 12, pp. 6106-6113, 2010) describe certain antibacterial activity of salicylanilides, however, there is no disclosure of the treatment of gonorrhea.

Ghazi et al. (Zentralbl. Mikrobiol. 141 (1986), 225-232) have tested the antibacterial effect and toxicity of synthesized salicylanilide derivatives against Escherichia coli, Bacillus subtilis, Pseudomonas aeruginosa and Staphylococcus aureus.

M. J. Macielag et al. tested for antibacterial activity of closantel and related derivatives against the drug-resistant organisms, methicillin-resistant Staphylococcus aureus (MRSA) and vancomycin-resistant Enterococcus faecium (VREF) (J. Med. Chem., 41(16), 2939-45 (1998)).

D. J. Hlasta et al. found that closantel had antibacterial activity against drug resistant S. aureus and E. faecium (Bioorg. Med. Chem. Letters, 8(14), 1923-28 (1998)).

R. Rajamuthiah et al. (PloS One, 2014, 9(2): e89189) identified closantel as a hit in a high throughput liquid screening assay and found anti-staphylococcal activity of closantel against vancomycin-resistant S. aureus isolates and other Gram-positive bacteria.

R. Rajamuthiah et al. (PloS One, 2015, 10(4):e0124595) describe that niclosamide and oxyclosanide have activity against MRSA.

Pauk et al. Bioorg. & Med. Chem. 23, 6574-6581 (2013), discloses the in-vitro anti-microbial activity of certain halogenated salicylanilides and derivatives.

WO 2008/155535 describes the use of halogenated salicylanilides for the treatment of acne resulting from propioni bacterial infection.

WO 2016/038035 relates to the use of closantel, rafoxanide, oxyclozanide, niclosamide and derivatives thereof in the topical treatment or prevention of infections caused by Gram-positive bacteria, such as Staphylococcus and Streptococcus.

WO 2016/193136 relates to the use of halogenated salicylanilides in the treatment of an infection caused by Clostridium bacteria, in particular Clostridium difficile.

WO 2016/080846 suggests treatment of an infection caused by Gram positive bacteria using a salicylamide compound and suggests also concurrent use of a salicylamide compound, such as nitazoxanide or niclosamide, and at least one efflux pump inhibitor for treatment of an infection caused by Gram negative bacteria. Examples of efflux pump inhibitor are phenylalanine-arginine β-napthylamide or 2,3-dibromomaleimide. Reference is made to U.S. Pat. No. 6,399,629 for further examples of efflux pump inhibitors.

WO 2017/200396 suggests concurrent use of at least one salicylamide compound, such as nitazoxanide or niclosamide, and an agent that increases the permeability of a bacterial cell membrane for treatment of an infection or for reducing or eliminating formation of a bacterial biofilm caused by Gram negative bacteria. Agents disclosed in WO 2017/200396 to increase the permeability of the bacterial cell membrane are polymyxin B, polymyxin E (colistin) and gramicidin. Polymyxins B and E are antibiotics used in the treatment of Gram-negative bacterial infections. Gramicidin is a heterogeneous mixture of three antibiotic compounds, gramicidins A, B and C, which are collectively called gramicidin D. Gramicidin has been found active against most Gram-positive bacteria and against some Gram-negative organisms, such as Neisseria bacteria.

WO 2016/036839 A1 suggests compositions and methods for preventing and treating gonorrhoea using a commensal (non-pathogen) species of Neisseria, such as Neisseria elongate and Neisseria polysaccharea.

### SUMMARY OF THE DISCLOSURE

It has been found that niclosamide, oxyclosanide, rafoxanide and closantel, in the absence of a bacterial efflux pump inhibitor and in the absence of an agent selected from the group consisting of polymyxins and gramicidins, are effective against Neisseria gonococcus, herein also referred to as N. gonococcus, and these compounds are therefore considered to be useful in treating, including preventing or reducing, an infection caused by N. gonococcus and possible reoccurrence of the infection in a subject.

In accordance with the present invention, there is provided a halogenated salicylanilide selected from the group consisting of closantel, rafoxanide, oxyclozanide, niclosamide, and pharmaceutically acceptable salts or solvates thereof, for use in the topical treatment, including prevention, of an infection caused by Neisseria gonococcus in a subject.

Use of halogenated salicylanilides, such as niclosamide, oxyclozanide, rafoxanide or closantel, may also reduce the rate of developing antibiotic resistance compared to known antibiotics used for the treatment of N. gonococcus infections.

In accordance with the present invention, there is provided a halogenated salicylanilide, such as closantel, rafoxanide, oxyclozanide, or niclosamide, or a pharmaceutically acceptable salt or solvate thereof, for use in the treatment, including prevention, of an infection caused by Neisseria gonococcus in a subject, wherein the subject is not treated concurrently with (i) a bacterial efflux pump inhibitor (e.g. phenylalanine-arginine β-napthylamide or 2,3-dibromomaleide) and/or (ii) an agent selected from the group consisting of polymyxins and gramicidins. In particular, the subject is not treated concurrently with (i) a bacterial efflux pump inhibitor (e.g. phenylalanine-arginine β-napthylamide or 2,3-dibromomaleide) and/or (ii) an agent that increase the permeability of a bacterial cell membrane, such as polymyxins and gramicidins.

Thus, there is provided a halogenated salicylanilide, such as closantel, rafoxanide, oxyclozanide, or niclosamide, or a pharmaceutically acceptable salt or solvate thereof, for use in the treatment, including prevention, of an infection caused by Neisseria gonococcus in a subject in the absence of concurrent use of a bacterial efflux pump inhibitor and in the absence of concurrent use of an agent selected from the group consisting of polymyxins and gramicidins.

The infection caused by N. gonococcus may be asymptomatic or sympotoamtic.

In embodiments, the infection by N. gonococcus results in a N. gonococcus associated infectious disease in the subject.

In embodiments, the N. gonococcus infection is an uncomplicated gonococcal infection or a disseminated gonococcal infection.

In embodiments, the N. gonococcus infection is (i) a urogenital, anorectal, pharyngeal and/or conjunctival gonococcal infection, or (ii) a disseminated gonococcal infection.

In embodiments, the N. gonococcus infection a urogenital, an anorectal, a pharyngeal or a conjunctival gonococcal infection.

In embodiments, the N. gonococcus infectious disease results from a combination of two or more gonococcal infections selected from a urogenital gonococcal infection, an anorectal gonococcal infection, a pharyngeal gonococcal infection or a conjunctival gonococcal infection.

In embodiments, the N. gonococcus infectious disease results from a disseminated gonococcal infection.

The disseminated gonococcal infection may cause one or more complications selected from skin lesions, tenosynovitis, perihepatitis, arthralgia, arthritis, arthritis-dermatitis syndrome, meningitis, or endocarditis.

In embodiments, the Neisseria gonococcus associated infectious disease is adult gonococcal conjunctivitis, pediatric gonococcal conjunctivitis or neonatal gonococcal conjunctivitis.

In embodiments, the Neisseria gonococcus associated infectious disease is neonatal gonococcal conjunctivitis.

In embodiments, the Neisseria gonococcus associated infectious disease is anorectal gonorrhoea, such as asymptomatic or symptomatic anorectal gonorrhoea.

In embodiments, the Neisseria gonococcus associated infectious disease is urogenital gonorrhoea, such as asymptomatic or symptomatic urogenital gonorrhoea.

In accordance with the present invention, there is also provided a halogenated salicylanilide, such as oxyclozanide, niclosamide, rafoxanide, or closantel, or a pharmaceutically acceptable salt or solvate thereof, for use in the topical treatment, including prevention, of conjunctival gonococcal infection, in particular neonatal gonococcal conjunctivitis.

In accordance with the present invention, there is also provided a halogenated salicylanilide, such as oxyclozanide, niclosamide, rafoxanide, or closantel, or a pharmaceutically acceptable salt or solvate thereof, for use in the topical treatment, including prevention, of anorectal gonococcal infection.

The halogenated salicylanilide may be used as the first line treatment of a N. gonococcus infection. By “first-line” treatment is meant the first treatment of the N. gonococcus. In the first line treatment, the N. gonococcus infection has not been treated with an antibiotic active against the N. gonococcus infection, for example, sulfonamides, penicillins, tetracyclines (e.g. doxycycline), quinolones (such as fluoroquinolones, e.g. ciprofloxacin, ofloxacin, and levofloxacin), cephalosporins (e.g. cephalexin, cefixime and ceftriaxone) or macrolides (e.g. azithromycin). Accordingly, it may be that the halogenated salicylanilide is for use in the treatment of a N. gonococcus infection, wherein the infection has not been treated with an antibiotic prior to administration of the halogenated salicylanilide to the subject.

The halogenated salicylanilide may be used to treat a recurrent N. gonococcus infection which has recurred following prior treatment of the subject with an antibiotic (or other agent) other than a halogenated salicylanilide. For example, the halogenated salicylanilide may be used to treat a N. gonococcus infection which has recurred in a subject following prior treatment of the subject with an antibiotic selected from sulfonamides, penicillins, tetracyclines (e.g. doxycycline), quinolones (such as fluoroquinolones, e.g. ciprofloxacin, ofloxacin, and levofloxacin), cephalosporins (e.g. cefixime and ceftriaxone) or macrolides (e.g. azithromycin).

The halogenated salicylanilide may be used to treat a N. gonococcus infection (for example a N. gonococcus infection) which is refractory (for example non-responsive) to treatment with an antibiotic (or other agent) other than a halogenated salicylanilide. For example, the halogenated salicylanilide may be used to treat a refractory N. gonococcus infection in a subject. Accordingly, the halogenated salicylanilide may be for use in the treatment of a N. gonococcus infection that is refractory to a prior antibiotic treatment other than a halogenated salicylanilide. For example the halogenated salicylanilide may be used to treat a N. gonococcus in a subject, wherein the N. gonococcus is refractory to treatment of the subject with an antibiotic selected from sulfonamides, penicillins, tetracyclines (e.g. doxycycline), quinolones (such as fluoroquinolones, e.g. ciprofloxacin, ofloxacin, and levofloxacin), cephalosporins (e.g. cephalexin, cefixime and ceftriaxone) or macrolides (e.g. azithromycin).

It may be that the halogenated salicylanilide is used to treat a N. gonococcus infection which is resistant to an antibiotic agent used to treat the N. gonococcus infection. Accordingly, there is provided a halogenated salicylanilide, or a pharmaceutically acceptable salt thereof, for use in the treatment of a N. gonococcus infection which is resistant to an antibiotic agent other than the halogenated salicylanilide.

It may be that the N. gonococcus is resistant to an antibiotic agent approved by the US FDA or European Medicines Agency prior to 2018, preferably an antibiotic approved for use in the treatment of a N. gonococcus infection. It may be that the N. gonococcus is resistant to an antibiotic selected from from sulfonamides, penicillins, tetracyclines (e.g. doxycycline), quinolones (such as fluoroquinolones, e.g. ciprofloxacin, ofloxacin, and levofloxacin), cephalosporins (e.g. cephalexin, cefixime and ceftriaxone) or macrolides (e.g. azithromycin). For example, it may be that the N. gonococcus is resistant to an antibiotic selected from ceftriaxone and/or azithromycin.

As illustrated in the Examples below, the single use of each of niclosamide, oxyclozanide, rafoxanide, and closantel, respectively, was unexpectedly found to be active against Neisseria gonococcus. Accordingly, it is expected that any of these compounds, or pharmaceutically acceptable salts or solvates thereof, will provide an effective treatment of N. gonococcus bacteria infections.

There is provided a halogenated salicylanilide, such as oxyclozanide, niclosamide, rafoxanide, or closantel, or a pharmaceutically acceptable salt or solvate thereof, for use in preventing or inhibiting transmission or spread of a N. gonococcus infection.

In particular, there is provided a halogenated salicylanilide, such as oxyclozanide, niclosamide, rafoxanide, or closantel, or a pharmaceutically acceptable salt or solvate thereof, for use, in the absence of concurrent use of a bacterial efflux pump and in the absence of concurrent use of an agent selected from the group consisting of polymyxins and gramicidins, in preventing or inhibiting transmission or spread of a N. gonococcus infection.

Also provided is a halogenated salicylanilide, such as oxyclozanide, niclosamide, rafoxanide, or closantel, or a pharmaceutically acceptable salt or solvate thereof, for use in a method of preventing or inhibiting transmission or spread of a N. gonococcus infection, the method comprising administering (e.g. orally or topically) an effective amount of the halogenated salicylanilide, or a pharmaceutically acceptable salt or solvate thereof, to a subject with a N. gonococcus infection. In particular, the subject is not administered concurrently with (i) a bacterial efflux pump inhibitor and/or (ii) an agent selected from the group consisting of polymyxins and gramicidins.

Also provided is a halogenated salicylanilide, such as oxyclozanide, niclosamide, rafoxanide, or closantel, or a pharmaceutically acceptable salt or solvate thereof, for use in a method of preventing or inhibiting the transmission or spread of a gonococcal infection from a mother to a infant, the method comprising topically administering an effective amount of a halogenated salicylanilide, or a pharmaceutically acceptable salt or solvate thereof, to the infant's eye to prevent a conjunctival gonococcal infection.

Also provided is a halogenated salicylanilide, such as oxyclozanide, niclosamide, rafoxanide, or closantel, or a pharmaceutically acceptable salt or solvate thereof, for use in a method of preventing or inhibiting the transmission or spread of a conjunctival gonococcal infection, the method comprising topically administering an effective amount of the halogenated salicylanilide, or a pharmaceutically acceptable salt or solvate thereof, to the subject with the conjunctival gonococcal infection.

Also provided is a halogenated salicylanilide, such as oxyclozanide, niclosamide, rafoxanide, or closantel, or a pharmaceutically acceptable salt or solvate thereof, for use in a method of preventing or inhibiting the transmission or spread of an anorectal gonococcal infection, the method comprising topically administering an effective amount of the halogenated salicylanilide, or a pharmaceutically acceptable salt or solvate thereof, to the subject with the anorectal gonococcal infection.

Also provided is a halogenated salicylanilide, such as oxyclozanide, niclosamide, rafoxanide, or closantel, or a pharmaceutically acceptable salt or solvate thereof, for use in a method of preventing or inhibiting recurrence of N. gonococcus infection in a subject with a N. gonococcus infection, the method comprising administering (e.g. orally or topically) an effective amount of the halogenated salicylanilide, or a pharmaceutically acceptable salt or solvate thereof, to the subject. In particular, the subject is not administered concurrently with (i) a bacterial efflux pump inhibitor and/or (ii) an agent selected from the group consisting of polymyxins and gramicidins.

Also provided is a halogenated salicylanilide, or a pharmaceutically acceptable salt or solvate thereof, for use in a method of preventing or inhibiting recurrence of conjunctival gonococcal infection in a subject, the method comprising topically administering an effective amount of the halogenated salicylanilide, or a pharmaceutically acceptable salt or solvate thereof, to the subject.

Also provided is a halogenated salicylanilide, or a pharmaceutically acceptable salt or solvate thereof, for use in a method of preventing or inhibiting recurrence of anorectal gonococcal infection in a subject, the method comprising topically administering an effective amount of the halogenated salicylanilide, or a pharmaceutically acceptable salt or solvate thereof, to the subject.

Halogenated Salicylanilides

Halogenated salicylanilides are also known as 2-hydroxy-N-phenylbenzamides or 2-hydroxybenzanilides. Salicylanilides are weakly acidic phenolic compounds. Halogenated salicylanilides are salicylanilides substituted by at least one halo group. The compounds were originally developed as fungicides for topical use and as antimicrobial agents in soaps. Later these compounds were shown to possess potent antihelmintic activity of which niclosamide, tribromosalan and clioxanide were some of the first agents to be used. A wide range of halogenated salicylanilide derivatives are known. Any halogenated salicylanilide possessing antibacterial activity against N. gonococcus may be used in the present invention. For example, the halogenated salicylanilide may be any of the niclosamide analogues described in WO 2008/021088, which are incorporated herein by reference thereto.

The halogenated salicylanilide may be a halogenated salicylanilide of the formula (I):

[chemical structure]

wherein
X is O or S;
R¹ and R² are at each occurrence independently selected from halo;
R³ and R⁴ are at each occurrence independently selected from H, C₁₋₆ alkyl, —OR^{A1}, —NO₂ and —CN;
R⁵ is H or -L¹-R⁷;
R⁶ is H or —C(O)R^{A2};
L¹ is selected from a bond, O, S, or —(CR^{A3}R^{B})ₒ—, wherein o is 1 or 2;
R⁷ is phenyl, unsubstituted or substituted with 1, 2, or 3 groups selected from halo, C₁₋₄ alkyl, —ORA⁴, —NO₂ and —CN;
RA¹, RA², RA³ and R^{A4} are at each occurrence independently selected from H and C₁₋₄ alkyl;
R^{B} is at each occurrence selected from H, C₁₋₄ alkyl and —CN;
n and p are each independently selected from 0, 1, 2, 3 or 4, with the proviso that n+p is at least 1;
t and v are independently selected from 0, 1 and 2;
or a pharmaceutically acceptable salt, or solvate thereof.

The halogenated salicylanilide of formula (I) may be of the formula (II), or a pharmaceutically acceptable salt, or solvate thereof.

[chemical structure]

The following statements in the numbered paragraphs below apply to compounds of the formulae (I) or (II). These statements are independent and interchangeable. In other words, any of the features described in any one of the following statements may (where chemically allowable) be combined with the features described in one or more other statements below. In particular, where a compound is exemplified or illustrated in this specification, any two or more of the statements below which describe a feature of that compound, expressed at any level of generality, may be combined so as to represent subject matter which is contemplated as forming part of the disclosure of this invention in this specification.
1. X is O.
2. R¹ and R² are at each occurrence independently selected from fluoro, chloro, bromo and iodo.
3. R¹ and R² are at each occurrence independently selected from chloro, bromo and iodo.
4. R¹ is chloro.
5. R¹ is bromo.
6. R¹ is iodo.
7. R² is chloro.
8. R² is bromo.
9. R² is iodo.
10. R³ and R⁴ are at each occurrence independently selected from H, C₁₋₄ alkyl, —OR^{A1}, —NO₂ and —CN.
11. R³ and R⁴ are at each occurrence independently selected from H, C₁₋₄ alkyl, —OR^{A1} and —NO₂.
12. R³ and R⁴ are at each occurrence independently selected from H, C₁₋₄ alkyl, —OH, —OMe, —NO₂ and —CN, for example H, C₁₋₄ alkyl, —OH or —NO₂.
13. R⁵ is H.
14. R⁵ is -L¹-R⁷.
15. L¹ is selected from —O—, —CH₂— and —CH(CN)—, for example —O— or —CH(CN)—.
16. R⁷ is phenyl, unsubstituted or substituted with 1, 2, or 3 groups selected from halo, C₁₋₄ alkyl and —CN
17. R⁷ is phenyl unsubstituted or substituted with 1, 2, or 3 groups (for example 1 or 2 groups) selected from halo.
18. R⁷ is unsubstituted phenyl.
19. L¹ is selected from —O— and —CH(CN)—; and R⁷ is phenyl unsubstituted or substituted with 1, 2, or 3 groups selected from halo.
20. R⁶ is H.
21. R⁶ is —C(O)R^{A2}, for example —C(O)CH₃.
22. t=0 or 1.
23. t=0.
24. v=0 or 1.
25. v=0.
26. o is 1.
27. v=1 and R⁴ is selected from —OH, C₁₋₄aalkyl and —NO₂.
28. A compound of any of formulae (I) or (II), or a pharmaceutically acceptable salt thereof.

Particular compounds are compounds of formula (I) or formula (II), or a pharmaceutically acceptable salt or solvate thereof wherein:
X is O;
R¹ and R² are at each occurrence independently selected from halo;
R³ and R⁴ are at each occurrence independently selected from H, C₁₋₄ alkyl, —OR^{A1}, —NO₂ and CN;
R⁵ is H or -L¹-R⁷;
R⁶ is H or —C(O)R^{A2};
L¹ is selected from O and —CH(CN)—;
R⁷ is phenyl unsubstituted or substituted with 1, 2, or 3 groups selected from halo;
R^{A1} and RA² are at each occurrence independently selected from H and C₁₋₄ alkyl;
n and p are each independently selected from 0, 1, 2, 3 or 4, with the proviso that n+p is at least 1;
t and v are independently selected from 0, 1 and 2.
or a pharmaceutically acceptable salt, or solvate thereof.

The halogenated salicylanilide may be selected from:

[chemical structure]

[chemical structure]

or a pharmaceutically acceptable salt or solvate thereof.

The halogenated salicylanilide may be a compound selected from Table 1 in WO 2008/021088, or a pharmaceutically acceptable salt thereof.

It may be that the halogenated salicylanilide, for example the halogenated salicylanilide of the formulae (I) or (II) is not the following compounds:

[chemical structure]

The halogenated salicylanilide may be selected from tetrachlorosalicylanilide, closantel, rafoxanide, oxyclozanide, resorantel, clioxanide, dibromosalan, tribromosalan, and niclosamide, or a pharmaceutically acceptable salt or solvate thereof.

The halogenated salicylanilide may be selected from tetrachlorosalicylanilide, closantel, rafoxanide, oxyclozanide, resorantel, clioxanide, dibromosalan, tribromosalan, and niclosamide, or a pharmaceutically acceptable salt thereof.

The halogenated salicylanilide may be selected from the group consisting of niclosamide, clioxanide, closantel, oxyclozanide, rafoxanide, tribromosalan, or a pharmaceutically acceptable salt or solvate thereof.

The halogenated salicylanilide may be selected from the group consisting of niclosamide, closantel, oxyclozanide, rafoxanide, or a pharmaceutically acceptable salt or solvate thereof.

The halogenated salicylanilide may be selected from the group consisting of niclosamide, closantel, oxyclozanide, rafoxanide, or a pharmaceutically acceptable salt thereof.

The halogenated salicylanilide may be closantel, or a pharmaceutically acceptable salt or solvate thereof, in particular the halogenated salicylanilide is closantel.

The halogenated salicylanilide may be rafoxanide, or a pharmaceutically acceptable salt or solvate thereof, in particular the halogenated salicylanilide is rafoxanide.

The halogenated salicylanilide may be selected from oxyclozanide, niclosamide, or a pharmaceutically acceptable salt or solvate thereof.

The halogenated salicylanilide may be niclosamide, or a pharmaceutically acceptable salt or solvate thereof, in particular the halogenated salicylanilide is niclosamide.

The halogenated salicylanilide may be oxyclozanide, or a pharmaceutically acceptable salt or solvate thereof, in particular the halogenated salicylanilide is oxyclozanide.

It is to be understood that any of the halogenated salicylanilides, in particular oxyclozanide, niclosamide, rafoxanide, or closantel, or a pharmaceutically acceptable salt or solvate thereof, described in this section or elsewhere in the application may be used in any of the treatments described herein.

The halogenated salicylanilide, such as oxyclozanide, niclosamide, rafoxanide, or closantel, or a pharmaceutically acceptable salt or solvate thereof, may be administered to the subject using any suitable route, for example parenterally (for example intravenous, intramuscular or subcutaneous administration), mucosal administration (for example oral or topical administration.

Suitably the halogenated salicylanilide is administered orally or topically.

More particularly, the halogenated salicylanilide is administered topically, such as via ocular, intravitreal, epicutaneous, intradermal, pharyngeal, anal, rectal, vaginal, penile or urethreal administration.

The halogenated salicylanilide may be topically administered to the subject using a topical formulation selected from a solution (including rinses, sprays, drops, e.g. eye drops), an emulsion, a suspension, a cream, a foam, a gel, a lotion, an ointment or a suppository, said topical formulation comprising the halogenated salicylanilide. The topical formulation may comprise the halogenated salicylanilide, or a pharmaceutically acceptable salt or solvate thereof, and a pharmaceutically acceptable excipient.

The halogenated salicylanilide may be topically administered to the subject using a device, such as a syringe, a condom or a vaginal pessary, coated or filled with the halogenated salicylanilide, or a pharmaceutically acceptable salt or solvate thereof. The halogenated salicylanilide, or a pharmaceutically acceptable salt or solvate thereof, may be present in a pharmaceutical composition, such as a topical formulation, comprising the halogenated salicylanilide or a pharmaceutically acceptable salt or solvate thereof.

The subject or patient in any of the treatments described is suitably a human or animal, for example a warm-blooded animal. Particularly the subject is a human.

Also provided is the use a halogenated salicylanilide, or a pharmaceutically acceptable salt or solvate thereof for the manufacture of a medicament for the treatment of an infection in a subject caused by N. gonococcus bacteria, said medicament lacking a bacterial efflux pump inhibitor and lacking an agent selected from the group consisting of polymyxins and gramicidins.

Also provided is a method of treating an infection caused by N. gonococcus bacteria in a subject, the method comprising administering (e.g. orally or topically) to said subject a therapeutically effective amount of a halogenated salicylanilide, or a pharmaceutically acceptable salt or solvate thereof. In particular, the subject is not administered concurrently with (i) a bacterial efflux pump inhibitor and/or (ii) an agent selected from the group consisting of polymyxins and gramicidins.

It is to be understood that the use and methods of the above two paragraphs are applicable to any of the infections, halogenated salicylanilides and routes of administration described herein.

Also provided is the use of a halogenated salicylanilide, or a pharmaceutically acceptable salt or solvate thereof, for the manufacture of a medicament for the topical treatment of conjunctival gonococcal infection, in particular neonatal gonococcal conjunctivitis, in a subject.

Also provided is the use of a halogenated salicylanilide, or a pharmaceutically acceptable salt or solvate thereof, for the manufacture of a medicament for the topical treatment of anorectal gonococcal infection in a subject.

Also provided is a method of treating a conjunctival gonococcal infection, in particular neonatal gonococcal conjunctivitis, in a subject, the method comprising topically administering to said subject an effective amount of a halogenated salicylanilide, or a pharmaceutically acceptable salt or solvate thereof.

Also provided is a method for preventing or inhibiting the transmission or spread of a Neisseria gonococcus infection from a mother to an infant, the method comprising topically administering an effective amount of a halogenated salicylanilide, or a pharmaceutically acceptable salt or solvate thereof, to the mother's vaginal tract to prevent a conjunctival gonococcal infection in the infant.

Also provided is a method for preventing or inhibiting the transmission or spread of a Neisseria gonococcus infection from a mother to an infant, the method comprising topically administering an effective amount of a halogenated salicylanilide, or a pharmaceutically acceptable salt or solvate thereof, to the infant's eye to prevent a conjunctival gonococcal infection.

Also provided is a method of treating an anorectal gonococcal infection in a subject, the method comprising topically administering to said subject an effective amount of a halogenated salicylanilide, or a pharmaceutically acceptable salt or solvate thereof.

Also provided is a condom coated with the halogenated salicylanilide, or a pharmaceutically acceptable salt or solvate thereof.

Also provided is a vaginal pessary coated with the halogenated salicylanilide, or a pharmaceutically acceptable salt or solvate thereof.

The halogenated salicylanilide, or a pharmaceutically acceptable salt or solvate thereof, may be comprised in a pharmaceutical composition when coated on the condom or vaginal pessary.

Also provided is a disposable syringe pre-filled with a topical formulation comprising the halogenated salicylanilide, or a pharmaceutically acceptable salt or solvate thereof. In embodiments, the topical formulation is selected from a solution, a suspension, an emulsion, a cream, a foam, a gel, a lotion or an ointment, in particular a solution, an emulsion, a suspension, a cream or a gel. The topical formulation may comprise the halogenated salicylanilide, or a pharmaceutically acceptable salt or solvate thereof, and a pharmaceutically acceptable excipient.

Preferred, suitable, and optional features of any one particular aspect of the present invention are also preferred, suitable, and optional features of any other aspect.

### DETAILED DESCRIPTION

### Definitions

Unless otherwise stated, the following terms used in the specification and claims have the following meanings set out below.

It is to be appreciated that references to “treating” or “treatment” include prophylaxis as well as the alleviation of established symptoms of a condition. Thus, “treatment” as used herein includes therapeutic (curative) treatment, prophylactic (preventing) treatment and palliative (alleviating) treatment of the indicated states, disorders or conditions. “Treating” or “treatment” of a state, disorder or condition therefore includes: (1) preventing or delaying the appearance of clinical symptoms of the state, disorder or condition developing in a subject, for example a human, that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms. Accordingly, in the context of treating infections caused by a N. gonococcus bacteria includes:

(i) the prevention of a disease caused by N. gonococcus;

(ii) the suppression of a disease caused by N. gonococcus;

(iii) the relief of symptoms of a disease caused by N. gonococcus;

(iv) the eradication of a non-symptomatic colonization by N. gonococcus species;

(v) the eradication of a N. gonococcus symptomatic infection;

(vii) the suppression of a disease caused a N. gonococcus infection from an area of the body affected by another noninfectious or infectious disease that enables establishment of an infection more readily, than in a non-disease affected area;

(viii) preventing or reducing the risk of transmission or spread of a N. gonococcus infection; or

(ix) preventing or reducing the risk of recurrence of a N. gonococcus infection.

As used herein, a “therapeutically effective amount” or “effective amount” means the amount of a compound that, when administered to a subject, for example a human, for treating a disease, is sufficient to effect such treatment for the disease. The “therapeutically effective amount” will vary depending on the compound, the disease and its severity and the age, weight, etc., of the subject to be treated.

Minimum inhibitory concentration (MIC) is the lowest concentration of an antibacterial that will inhibit the visible growth of a microorganism after overnight incubation. Minimum inhibitory concentrations are important in diagnostic laboratories to confirm resistance of microorganisms to an antimicrobial agent and also to monitor the activity of new antimicrobial agents. A MIC is generally regarded as the most basic laboratory measurement of the activity of an antimicrobial agent against an organism.

The median lethal dose, LD50 (abbreviation for “lethal dose, 50%”) of a toxin, radiation, or pathogen is the dose required to kill half the members of a tested population after a specified test duration. LD50 figures are frequently used as a general indicator of a substance's acute toxicity.

Therapeutic index (therapeutic ratio) is defined as the amount of a therapeutic agent causing the therapeutic effect measured as MIC to the amount that causes death in animal studies measured as LD50.

The rate of resistance development is quantified as the frequency of spontaneous mutants in a population of bacteria that is able to resist a given concentration of an antibiotic. For example, the rate of resistance development may be 10⁻⁹ if on average 1 cell in 10⁹ cells is able to survive a concentration of antibiotic corresponding to 1× MIC incubated at 37° C. for 48 hours using the method described in Drago et al. Journal of Antimicrobial Chemotherapy, 2005, 56(2), 353 to 359).

In microbiology, colony-forming unit (CFU) is an estimate of the number of viable bacteria or fungal cells in a sample. Viable is defined as the ability to multiply via binary fission under the controlled conditions.

The term “halo” or “halogen” refers to one of the halogens, group 17 of the periodic table. In particular, the term refers to fluorine, chlorine, bromine and iodine.

The term Cₘ-Cₙ refers to a group with m to n carbon atoms.

The term “C₁-C₆alkyl” refers to a linear or branched hydrocarbon chain containing 1, 2, 3, 4, 5 or 6 carbon atoms, for example methyl, ethyl, n-propyl, iso-propyl, n-butyl, sec-butyl, tert-butyl, n-pentyl and n-hexyl. “C₁-C₄alkyl” similarly refers to such groups containing up to 4 carbon atoms.

The term “optionally substituted” refers to either groups, structures, or molecules that are substituted and those that are not substituted.

Where optional substituents are chosen from “one or more” groups it is to be understood that this definition includes all substituents being chosen from one of the specified groups or the substituents being chosen from two or more of the specified groups.

Where a moiety is substituted, it may be substituted at any point on the moiety where chemically possible and consistent with atomic valency requirements. The moiety may be substituted by one or more substituents, e.g. 1, 2, 3 or 4 substituents; optionally there are 1 or 2 substituents on a group. Where there are two or more substituents, the substituents may be the same or different.

Substituents are only present at positions where they are chemically possible, the person skilled in the art being able to decide (either experimentally or theoretically) without undue effort which substitutions are chemically possible and which are not.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

Suitable or preferred features of any compounds of the present invention may also be suitable features of any other aspect.

The invention contemplates pharmaceutically acceptable salts of the halogenated salicylanilide compounds of the invention. These may include the acid addition and base salts of the compounds. These may be acid addition and base salts of the compounds. Suitable acid addition salts are formed from acids which form non-toxic salts. Suitable base salts are formed from bases which form non-toxic salts.

Pharmaceutically acceptable salts of the halogenated salicylanilide compounds may be prepared by for example, one or more of the following methods:
(i) by reacting the compound of the invention with the desired acid or base; or
(ii) by converting one salt of the compound of the invention to another by reaction with an appropriate acid or base or by means of a suitable ion exchange column.

These methods are typically carried out in solution. The resulting salt may precipitate out and be collected by filtration or may be recovered by evaporation of the solvent. The degree of ionisation in the resulting salt may vary from completely ionised to almost non-ionised.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”. Where a compound of the invention has two or more stereocentres any combination of (R) and (S) stereoisomers is contemplated. The combination of (R) and (S) stereoisomers may result in a diastereomeric mixture or a single diastereoisomer. The compounds of the invention may be present as a single stereoisomer or may be mixtures of stereoisomers, for example racemic mixtures and other enantiomeric mixtures, and diasteroemeric mixtures. Where the mixture is a mixture of enantiomers the enantiomeric excess may be any of those disclosed above. Where the compound is a single stereoisomer the compounds may still contain other diasteroisomers or enantiomers as impurities. Hence a single stereoisomer does not necessarily have an enantiomeric excess (e.e.) or diastereomeric excess (d.e.) of 100% but could have an e.e. or d.e. of about at least 85%

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E- and Z-isomers). It is to be understood that the present invention encompasses all optical, diastereoisomers and geometric isomers and mixtures thereof that possess activity against N. gonococcus bacteria.

It is also to be understood that certain compounds of the invention, or salts or solvates thereof, may exist in solvated as well as unsolvated forms such as, for example, hydrated forms. It is to be understood that the invention encompasses all such solvated forms that possess activity against N. gonococcus bacteria.

It is also to be understood that the halogenated salicylanilides of the invention may exhibit polymorphism, and that the invention encompasses all such forms that possess activity against N. gonococcus bacteria.

It is further to be understood that the halogenated salicylanilide may be used in the form of suitable pharmaceutically-acceptable pro-drug of the compound and that such prodrugs are intended to be encompassed by the invention. Accordingly, halogenated salicylanilide may be administered in the form of a pro-drug, that is a compound that is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable solvate derivatives that may be formed at a hydroxy group in a compound.

Accordingly, the present invention includes the halogenated salicylanilides as defined hereinbefore when made available by organic synthesis and when made available within the human or animal body by way of cleavage of a pro-drug thereof. Accordingly, the present invention includes those halogenated salicylanilide compounds that are produced by organic synthetic means and also such compounds that are produced in the human or animal body by way of metabolism of a precursor compound, that is the halogenated salicylanilide may be a synthetically-produced compound or a metabolically-produced compound.

A suitable pharmaceutically-acceptable pro-drug of a halogenated salicylanilide compound is one that is based on reasonable medical judgement as being suitable for administration to the human or animal body without undesirable pharmacological activities and without undue toxicity.

Various forms of pro-drug have been described, for example in the following documents:

a) Methods in Enzymology, Vol. 42, p. 309-396, edited by K. Widder, et al. (Academic Press, 1985);

b) Design of Pro-drugs, edited by H. Bundgaard, (Elsevier, 1985);

c) A Textbook of Drug Design and Development, edited by Krogsgaard-Larsen and H. Bundgaard, Chapter 5 “Design and Application of Pro-drugs”, by H. Bundgaard p. 113-191 (1991);

d) H. Bundgaard, Advanced Drug Delivery Reviews, 8, 1-38 (1992);

e) H. Bundgaard, et al., Journal of Pharmaceutical Sciences, 77, 285 (1988);

f) N. Kakeya, et al., Chem. Pharm. Bull., 32, 692 (1984);

g) T. Higuchi and V. Stella, “Pro-Drugs as Novel Delivery Systems”, A.C.S. Symposium Series, Volume 14; and

h) E. Roche (editor), “Bioreversible Carriers in Drug Design”, Pergamon Press, 1987.

The halogenated salicylanilide may be used in the form of a prodrug of the compound for example, an in vivo cleavable solvate thereof. An in vivo cleavable solvate of a compound may be, for example, a pharmaceutically-acceptable solvate which is cleaved in the human or animal body to produce the parent compound.

A suitable pharmaceutically-acceptable pro-drug of a halogenated salicylanilide that possesses a hydroxy group is, for example, an in vivo cleavable solvate or ether thereof. An in vivo cleavable ester or ether of a compound containing a hydroxy group is, for example, a pharmaceutically-acceptable ester or ether which is cleaved in the human or animal body to produce the parent hydroxy compound. Suitable pharmaceutically-acceptable ester forming groups for a hydroxy group include inorganic esters such as phosphate esters (including phosphoramidic cyclic esters). Further suitable pharmaceutically-acceptable ester forming groups for a hydroxy group include C₁₋₁₀alkanoyl groups such as acetyl, benzoyl, phenylacetyl and substituted benzoyl and phenylacetyl groups, C₁₋₁₀alkoxycarbonyl groups such as ethoxycarbonyl, N,N—(C₁₋₆)₂carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C₁₋₄alkyl)piperazin-1-ylmethyl. Suitable pharmaceutically-acceptable ether forming groups for a hydroxy group include α-acyloxyalkyl groups such as acetoxymethyl and pivaloyloxymethyl groups. Accordingly reference to a “pharmaceutically acceptable ester” of a compound encompasses the esters described above.

### Halogenated Salicylanilides

The halogenated salicylanilide used in the present invention may be any of the halogenated salicylanilides described herein, or a pharmaceutically acceptable salt thereof, or a pharmaceutically acceptable solvate thereof, or a pro-drug of any thereof.

Particular halogenated salicylanilides include the compounds of formulae (I) and (II) or a pharmaceutically acceptable salt thereof as described herein.

More particularly the halogenated salicylanilide is selected from the group consisting of tetrachlorosalicylanilide, closantel, rafoxanide, oxyclozanide, resorantel, clioxanide, dibromosalan, tribromosalan and niclosamide. Even more particularly, the halogenated salicylanilide is selected from the group consisting of closantel, rafoxanide, oxyclozanide, and niclosamide.

Niclosamide

In a one embodiment of the invention the halogenated salicylanilide is niclosamide or a pharmaceutically acceptable salt thereof. Niclosamide (2′,5-dichloro-4′-nitrosalicylanilide) exhibits the following acute toxicity:

LD₅₀, mice, p.o., >5000 mg/kg

LD₅₀, rats, p.o., 5000 mg/kg

LD₅₀, rats, dermal, 2000 mg/kg

LD₅₀, rabbits, p.o., 5000 mg/kg

LD₅₀, cats, p.o., >1000 mg/kg

Niclosamide thus exhibits low toxicity. The compound is poorly soluble in water and shows low intestinal absorption. Once in the bloodstream niclosamide is quickly cleared via the urinary tract or by enzymatic metabolism in the liver.

### Niclosamide Derivatives

Illustrative niclosamide analogs include, but are not limited to closantel (CAS #: 57808-65-8), oxyclozanide (CAS #: 2277-92-1), rafoxanide (CAS #: 22662-39-1), clioxanide (CAS #: 14437-41-3). Other suitable niclosamide analogs include, 4′-chloro-3-nitrosalicylanilide, 4′-chloro-5-nitrosalicylanilide, 2′-chloro-5′-methoxy-3-nitro-salicylanilide, 2′-methoxy-3,4′-dinitrosalicylanilide, 2′,4′-dimethyl-3-nitrosalicylanilide, 2′-ethyl-3-nitrosalicylanilide and 2′-bromo-3-nitrosalicyl-anilide; or a pharmaceutically acceptable salt thereof. Further niclosamide derivatives include those described in WO 2008/021088, particularly those described in Table 1 therein, which are incorporated herein by reference.

Particular niclosamide analogues include closantel, rafoxanide and oxyclozanide. These compounds are expected to have a suitable toxicity profile for the use described herein.

Acute toxicity of closantel:

LD₅₀, rats, p.o., 262-342 mg/kg (depending on the study), median 302 mg/kg

LD₅₀, rats, s.c., 67 mg/kg

LD₅₀, mice, p.o., 331 mg/kg

LD₅₀, mice, i.m., 57 mg/kg

Acute toxicity of rafoxanide:

LD₅₀, rats, p.o., 980->2000 mg/kg (depending on the study), median >1490 mg/kg

LD₅₀, mice, p.o., 232-300 mg/kg (depending on the study), median 266 mg/kg

LD₅₀, rabbits, p.o., 3200 mg/kg

Acute toxicity of oxyclozanide.

LD₅₀, rats, p.o., 980-3519 mg/kg (depending on the study), median 2250 mg/kg

LD₅₀, mice, p.o., 300 mg/kg

LD₅₀, rabbits, p.o., 3200 mg/kg

Particularly niclosamide or oxyclozanide, or a pharmaceutically acceptable salt or solvate thereof, is believed to be useful for treatment of infections caused by Neisseria gonococcus.

### Brominated Halogenated Salicylanilides

In another embodiment, the halogenated salicylanilide is a brominated halogenated salicylanilide, for example 4′,5-dibromosalicylanilide (also known as dibromsalan); 3,5-dibromosalicylanilide (also known as metabromsalan; and 3,4′,5-tribromosalicylanilide (also known as tribromsalan).

Synthesis

The halogenated salicylanilides described herein are known or can be synthesised using known methods. For example, using methods analogous to those described in WO2004/006906. The compounds of the Formula (I) herein may be prepared by coupling an amine of the formula (III) with an acid of formula (IV):

[chemical structure]

Necessary starting materials are known or can be prepared using standard procedures of organic chemistry.

### Bacterial Efflux Pump Inhibitor

As mentioned above, WO 2016/080846 discloses certain salicylamide compounds and at least one efflux pump inhibitor for treatment of an infection caused by Gram negative bacteria. WO 2016/080846 discloses the following classes of efflux pump compounds:

alkoxyquinoline derivatives, e.g. 2,8-dimethyl-4-(2′-pyrrolidinoethyl)-oxyquinoline;

piperidine and piperidine analogues;

phenothiazines, e.g. chloropromazine;

monoterpene derivatives, e.g. geranylamine; and

arginine derivatives (see U.S. Pat. No. 6,251,869);

The following compounds are disclosed in WO 2016/080846 as examples of bacterial efflux pump inhibitors: (2R,4S)-4-(2-aminoacetamido)-N-[(1R-)3-phenyl-1-(3-quinolylcarbamoyl)propyl]-2-pyrrolidinecarboxamide; (2R,4S)-4-(2-aminoacetamido)-N-[(1R)-3-phenyl-1-(3-phenyl)propylcarbamoyl]propyl]-2-pyrrolidinecarboxamide; (2S,4R)-4-(2-aminoacetamido)-N-[(1R)-3-phenyl-1-(3-quinolylcarbamoyl)propyl]-2-pyrrolidinecarboxamide; (2S,4R)-4-(2-aminopropionamido)-N-[(1R)-3-phenyl-1-(3-quinolylcarbamoyl)propyl]-2-pyrrolidinecarboxamide; (2S,4R)-4-(aminompropionamido)-N-[(1R)-3-phenyl-1-(3-quinolylcarbamolyl)propyl]-2-pyrrolidinecarboxamide; (2S,4R)-4-[3-(aminopropionamido])-N-[(1R)-3-phenyl-1-(3-quinolylcarbamoyl)propyl]-2-pyrrolidinecarboxamide; (2S,4R)-4-(amino-N-[(1R)-3-pheynl-1-(3-quinolylcarbamoyl)propyl]-2-pyrrolidinecarboxamide; (2S,4R)-4-(aminoacetamido)-N-[(1R)-3-methyl-1-[(3-quinolylcarbamoyl)butyl]-2-pyrrolidinecarboxamide; (2S,4S-4-(2-amino-N-methylacetamido)-N-[(1R)-3-phenyl-1-(3-quinolylcarbamoyl)propyl]-2-pyrrolidinecarboxamide; (2S,4R)-4-(2-aminoacetamido)-N-[(1R)-3-3-methyl-1-(6-methoxy-8-methyl-3-quinolylcarbamoyl)butyl]-2-pyrrolidinecarboxamide; (2S,4R)-4-(2-aminoacetamido)-N-[(1R)-3-phenyl-1-(6,7-dimethyl-3-quinolylcarbamoyl)propyl]-2-pyrrolidinecarboxamide; (2S,4R)-4-(2-aminoacetamido)-N-[(1R)-2-(4-methoxyphenyl)-1-(3-quinolylcarbamoyl)ethyl]-2-pyrrolidinecarboxamide; (2R,4R)-4-(aminomethyl)-N-[(1R)-3-methyl-1-quinoylcarbamoyl)bulyl]-2-pyrrolidinecarboxamide; (2R,4R)-4-aminomethyl)-N-[(1R)-3-phenyl-1-(6-ethyl-3-quinolylcarbamoyl)propyl]-2-pyrrolidinecarboxamide; (2S,4R)-4-(2-aminoacetamido)-N-methyl-N-[(1R)-3-phenyl-1-(3-quinolylcarbamoyl)propyl]-2-pyrrolidinecarboxamide; (2R,4R)-4-(aminomethyl)-N-[(1R)-3-phenyl-1-(3-quinolylcarbamoyl)propyl]-2-pyrrolidinecarboxamide; (2R,4R)-4-(2-aminoacetamido)-N-[(1R)-3-phenyl-1-(3-quinolylcarbamoyl)propyl]-2-pyrrolidinecarboxamide; (2S,4R)-4-(2-aminoacetamido)-N-[(1R)-3-phenyl-1-(6-ethyl-3-quinolylcarbarrioyl)propyl]-2-pyrrolidinecarboxamide; (2S,4R)-4-(2-aminoacetamido)-N-[(1R)-3-methyl-1-(6-ethyl-3-quinolylcarbamoyl)butyl]-2-pyrrolidinecarboxamide; (2R,4R)-4-(2-aminoacetamido)-N-[(1R)-2-(4-fluorophenyl)-1-(3-quinolylcarbamoyl)ethyl]-2-pyrrolidinecarboxamide; (2S,4R)-4-(2-aminoacetamido)-N-[(1R)-2-(4-fluorophenyl)-1-(3-quinolylcarbamoyl)ethyl]-2-pyrrolidnecarboxamide; (2S,4R)-1-(2-aminoacetamido)-N-[(1R)-3-phenyl-1-(6-methoxy-8-methyl-3-quinolylcarbamoyl)propyl]2-pyrrolidinecarboxamide; (2S,4R)-4-(2-aminoacetamido)-N-[1R)-2-(4-hydroxyphenyl)-1-(3-quinolylcarbamoyl)ethyl]-2-pyrrolidinecarboxamide; (2S,4S)-4-(aminomethyl)-N-[(1R)-3-pheny[-1-(3-quinolylcarbamoyl)propyl]-2-pyrrolidinecarboxamide; (2R,4R)-4-(aminomethyl)-N-[(1R)-2-(4-hydroxyphenyl)-1-(6-ethyl-3-quinolylcarbamoyl)ethyl]-2-pyrrolidinecarboxamide; (2S,4R)-4-(2-aminoacetamido)-N-[(1R)-3-phenyl-1-(5-chloro-2-hydroxyphenylcarbamoyl)propyl]-2-pyrrolidinecarboxamide; (2S,4R)-4-(2-aminoacetamido)-N-[(1R)-3-phenyl-1-(4,5-dimethyl-2-hydroxyphenylcarbamoyl)propyl]-2-pyrrolidinecarboxamide; (2S,4R)-4-(2-aminoacetamido)-N-[(1R)-3-phenyl-1-(2-hydroxy-5-methylphenylcarbamoyl)propyl]-2-pyrrolidinecarboxamide; (2R,4R)-4-(aminomethyl)-N-[(1R)-3-phenyl-1-(6-quinolylcarbamoyl)propyl]-2-pyrrolidinecarboxamide; (2R,4R)-4-(aminomethyl)-N-[(1R)-3-phenyl-1-(8-quinolylcarbamoy propyl]-2-pyrrolidinecarboxamide; (2R,4R)-4-(aminomethyl)-N-[(1R)-2-(4-fluoropheny)-1-(3-quinolylcarbamoyl)ethyl]-2-pyrrolidinecarboxamide; (2R,4R)-4-(aminomethyl)-N-[(1R)-3-phenyl-1-[3-quinolylmethyl)carbamoyl]propyl]-2-pyrrolidinecarboxamide; (2R,4R)-4-(aminomethyl)-N-methyl-N-[(1R)-3-phenyl-1-(3-quinolylcarbamoyl)propyl]-2-pyrrolidinecarboxamide; (2R,4R)-4-(aminomethyl)-N-[(1R)-3-methyl-1-(3-quinolylcarbamoly)butyl]-2-pyrrolidinecarboxamide; (2 R,4R)-4-(aminomethyl)-N-[(1R)-3-phenyl-1-(6-fluoro-3-quinolylcarbamoyl)propyl]-2-pyrrolidinecarboxamide; (2R,4R)-4-(aminomethyl)-N-[(1R)-4-methyl-1-(3-quinolylcarbamoyl)pentyl]-2-pyrrolidinecarboxamide; (2R,4R)-4-(aminomethyl)-N-[(1R)-3-phenyl-1-(5-fluoro-2-hydroxyphenylcarbamoyl)propyl]-2-pyrrolidinecarboxamide; (2 R,4R)-4-(aminomethyl)-N-[(1R)-3-phenyl-1-(2-hydroxy-5-methylphenylcarbamoyl)propyl]-2-pyrrolidinecarboxamide; (2R,4R)-4-(aminomethyl)-N-[(1R)-3-phenyl-1-(5-chloro-2-hydroxyphenylcarbamoyl)propyl]-2-pyrrolidinecarboxamide; (2R,4R)-4-(aminomethyl)-N-methyl-N-[(1R)-3-methyl-1-(6-ethyl-3-quinolylcarbamoyl)butyl]-2-pyrrolidinecarboxamide; (2R,4S)-4-(2-aminoacetamido)-N-(2-phenylethyl)-N-(3-quinolylcarbamoyl)ethyl]-2-pyrrolidinecarboxamide; (2 R,4S)-4-[(2R)-2-aminopropionamido]-N-(2-phenylethyl)-N-(3-quinolylcarbamoyl)ethyl]-2-pyrrolidinecarboxamide; (2S,4R)-4-(2-aminoacetamido)-N-(2-phenylethyl)-N-(3-quinolylcarbamoyl)ethyl]-2-pyrrolidinecarboxamide; (2 R,4S)-4-(2-aminoacetamido)-N-(2-methylpropyl)-N-(7-ethyl-3-quinolylcarbamoyl)ethyl]-2-pyrrolidinecarboxamide; (2R,4R)-4-(aminomethyl)-N-(2-phenylethyl)-N-(3-quinolylcarbamoyl)ethyl]-2-pyrrolidinecarboxamide; (2 R,4R)-4-(2-aminoacetamido)-N-(2-phenylethyl)-N-(3-quinolylcarbamoyl)methyl]-2-pyrrolidinecarboxamid; (2R,4S)-4-[(2R)-2-aminopropionamido]-N-(3,3-dimethylbutyl)-N-(3-quinolylcarbamoyl)methyl]-2-pyrrolidinecarboxamide; (2 R,4R)-4-(aminomethyl)-N-[(1R)-3-phenyl-1-[(2-quinolyloxy)methyl]propyl]-2-pyrrolidinecarboxamide; (2R,4S)-4-(2-aminoacetamido)-N-[(1R)-3-methyl-1-[(2-naphthyloxy)methyl]butyl]-2-pyrrolidinecarboxamide; (2S,4R)-4-(2-aminoacetamido)-N-[(1R)-3-phenyl-1-[(4-chlorophenylthio)methyl]propyl]-2-pyrrolidinecarboxamide; (2S,4R)-4-[(2R)-2-aminopropionamido]-N-[(1R)-3-phenyl-1-[(4-chlorophenylthio)methyl]propyl]-2-pyrrolidinecarboxamide; (2R,4S)-4-[(2R)-2-aminopropionamido]-N-[(1R)-3-phenyl-1-[(4-chlorophenylthio)methyl]propyl]-2-pyrrolidinecarboxamide; (2S,4R)-4-(2-aminoacetamido)-N-[(1R)-3-phenyl-1-[(3-quinolylthio)methyl]propyl]-2-pyrrolidinecarboxamide; (2S,4R)-4-(2-aminoacetamido)-N-[(1R)-3-phenyl-1-[(2-quinolyloxy)methyl]propyl]-2-pyrrolidinecarboxamide; (2S ,4 R)-4-(2-aminoacetamido)-N-[(1R)-3-phenyl-1-[(2-quinolylthio)methyl]propyl]-2-pyrrolidinecarobxamide; (2S,4R)-4-(2-aminoacetamido)-N-[(1R)-3-phenyl-1-(phenylthiomethyl)propyl]-2-pyrrolidinecarboxamide; (2S,4R)-4-(2-aminoacetamido)-N-[(1R)-3-phenyl-1-[(4-fluorophenylthio)methyl]propyl]-2-pyrrolidinecarboxamide; (2S,4R)-4-(2-aminoacetamido)-N-[(1R)-3-methyl-1-[(2-quinolyloxy)methyl]butyl]-2-pyrrolidinecarboxamide; (2R,4R)-4-(aminomethyl)-N-[(1R)-3-methyl-1-[(3-quinolyloxy)methyl]buty[]-2-pyrrolidinecarboxamide; (2S,4R)-4-(2-aminoacetamido)-N-[(1R)-3-methyl-1-[(4-chlorophenylthio)methyl]butyl]-2-pyrrolidinecarboxamide; (2R,4R)-4-(aminomethyl)-N-[(1R)-3-methyl-1-[(4-chlorophenylthio)methyl]butyl]-2-pyrrolidinecarboxamide; (2R,4R)-4-(aminomethyl)-N-[(2S)-2-(6-methyl-3-quinolylcarboxamido)-4-phenylbutyl]-2-pyrrolidinecarboxamide; (2R,4R)-4-(aminomethyl)-N-[(1R)-3-phenyl-1-[(6-methyl-3-quinolylcarboxamido)methyl]propyl]-2-pyrrolidinecarboxamide; (2S,4R)-4-(2-aminoacetamido)-N-[(1R)-1-[(RS)-(5,6-dimethyl-2-benzoxazolyl)hydroxymethyl]-3-phenylpropyl]-2-pyrrolidinecarboxamide; (2S,4R)-4-[(2R)-2-aminopropionamido]-N-[(1R)-1-[(RS)-(5,6-dimethyl-2-benzoxazolyl)hydroxymethyl]-3-phenylpropyl]-2-pyrrolidinecarboxamide; (2R,4S)-4-[(2R)-2-aminopropionamido]-N-[(1R)-1-[(RS)-(5,6-dimethyl-2-benzoxazolyl)hydroxymethyl]-3-phenylpropyl]-2-pyrrolidinecarboxamide; (2S,4R)-4-(2-aminoacetamido)-N-[(1R)-1-[5,6-dimethyl-2-benzoxazolyl)hydroxymethyl]-3-phenylpropyl]-2-pyrrolidinecarboxamide; (2R,4R)-4-(aminomethyl)-N-[(1R)-1-[(RS)-(5-1,1-dimethyl)ethyl-2-benzoxazolyl)hydroxymethyl]-3-phenylpropyl]-2-pyrrolidinecarboxamide; (2R,4R)-4-(aminomethyl)-2-[((1S)-3-phenyl-1-(3-quinolylcarbamoyl)propyl)oxymethyl]-pyrrolidine; (2R,4R)-4-(aminomethyl)-2-[((1R)-3-phenyl-1-(3-quinolylcarbamoyl)propyl)-oxymethyl]pyrrolidine; (2R,4R)-4-(aminomethyl)-2-(2-quinolyloxymethyl)pyrrolidine; (2R,4R)-4-(aminomethyl)-2-(6-methyl-3-quinolylcarboxamidomethyl)pyrrolidine; (2S,4R)-4-(2-aminoacetamido)-2-(5-benzyl-2-benzmidazolyl)pyrrolidine; (2R,4R)-4-(aminomethyl)-2-(5-benzyl-2-benzimidazolyl)pyrrolidine; (2S,4R)-4-(2-aminoacetamido)-2-(1-(2-phenyl)ethyl-2-benzimidazolyl)pyrrolidine; (2S,4R)-4-(2-aminoacetamido)-2-(1-(3-aminopropyl)-2-benzimidazolyl)pyrrolidine; (2S,4R)-4-(2-aminoacetamido)-N-[(1R)-1-(2-benzoxazolyl)-3-phenylpropyl]-2-pyrrolidinecarboxamide; (2S,4R)-4-(2-aminoacetamido-N-[(1S)-1-(2-benzimidazolyl)-3-phenylpropyl]-2-pyrrolidinecarboxamide; (2S,4R)-4-(2-am inoacetamido)-N-[(5-benzyl-2-benzimidazolyl)methyl]-2-pyrrolidinecarboxamide; (2S,4R)-4-(2-aminoacetamido)-N-[(1-(2-phenypethyl-2-benzimidazolyl)methyl]-2-pyrrolidinexarboxamide; (2S,4R)-4-(2-aminoacetamido)-N-[(5-1 ,1-dimethyl)ethyl-2-benzimidazolyl)methyl]-2-pyrrolidinecarboxamide; (2S,4R)-4-(2-aminoacetamido)-N-[(5-(1 hydroxy-1-phenyl)methyl-2-benzimidazolyl)methyl]-2-pyrrolidinecarboxaniide; (2S,4R)-4-(2-aminoacetamido)-N-[(1 S)-1-(5-benzyl-2-benzimidazolyl)ethyl]-2-pyrrolidinecarboxamide; (2S ,4R)-4-(2-aminoacetamido)-N-[(1R)-1-(2-benzthiazolyl)-3-phenylpropyl]-2-pyrrolidinecarboxamide; (2S,4R)-4-(2-aminoacetamido)-N-[(1S)-1-(2-benzoxazolyl)-3-phenylpropyl]-2-pyrrolidinecarboxamide; (2R,4R)-4-(aminomethyl)-N-[(5-benzyl-2-benzimidazolyl)methyl]-2-pyrrolidinecarboxamide; (2R,4S)-4-(aminomethyl)-N-[(5-benzyl-2-benzimidazolyl)methyi]-2-pyrrolidinecarboxamide; (2R,4R)-4-(aminomethyl)-N-[(5-phenyloxy-2-benzimidazolyl)methyl]-2-pyrrolidinecarboxamide; (2R,4R)-4-(aminomethyl)-N-[(5-phenyl-2-benzimidaxolyl)methyl]-2-pyrrolidinecarboxamide; (2S,4R)-4-(2-aminoethylthio)-N-[(1R)-3-phenyl-1-(3-quinolylcarbamoyl)propyl]-2-pyrrolidinecarboxamide; (2S,4R)-4-(2-aminoethyloxy)-N-[(1R)-3-phenyl-1-(3-quinolylcarbamoyl)propyl]-2-pyrrolidinecarboxamide; (2S,4R)-4-(2-aminoethyloxy)-N-(6-(1,1-dimethyl)ethyl-3-quinolyl)-2-pyrrolidinecarboxamide; (2S,4RS)-4-(3-aminopropyl)-N-[(1R)-3-phenyl-1-(3-quinolylcarbamoyl)propyl]-2-pyrrolidinecarboxamide; (2S,4R)-4-(2-aminoacetamido)-N-[5-(p-chlorophenyl)tetrahydro-3-thienyl]-2-pyrrolidinecarboxamide; (2S,4R)-4-(guanadinyl)-N-[(1R)-3-phenyl-1-(3-quinolylcarbamoyl)propyl]-2-pyrrolidinecarboxamide; (2S,4R)-4-(2-aminoacetamido)-N-[7-ethyl-3-quinolyl]-2-pyrrolidinecarboxamide; (2R,4R)-4-(aminomethyl)-N-[6-(l,l-dimethyl)ethyl-3-quinolyl]-2-pyrrolidinecarboxamide; (2S,4R)-4-(2-aminoacetamido)-N-(5-benzyl-2-hydroxyphenyl)-2-pyrrolidinecarboxamide; (2S,4R)-4-(2-aminoacetamido)-N-[4-benzyl-2-benzimidazolyl)ethyl]-2-pyrrolidinecarboxamide; (2R,4R)-4-(aminomethyl)-N-(6-ethyl-3-quinolyl)-2-pyrrolidinecarboxamide; (2R,4R)-4-(aminomethyl)-N-(5-benzyl-2-benzimidazolyl)-2-pyrrolidinecarboxamide; (2S,4R)-4-(2-aminoacetamido)-N-(5-benzyl-2-benzimidazolyl)-2-pyrrolidinecarboxamide; (2R,4R)-4-(aminomethyl)-N-[(1R)-3-phenyl-1-[(3-quinolylcarboxamido)methyl]propyl]-2-pyrrolidinecarboxamide; trans-4-glycylamino-D-prolyl-D-proline-(6-isopropyl)-3-quinolylamide; trans-4-amino-L-prolyl-trans-4-((R)-2-hydroxy-4-phenylbutyrylamino-L-proline 3-quinolylamide; trans-4-glycylamino-L-prolyl-trans-4-((R)-2-hydroxy-4-phenylbutrylamino)-L-proline 3-quinolylamide; cis-4-glycylamino-L-prolyl-trans-4-((R)-2-hydroxy-4-phenylbutyrylamino)-L-proline 3-quinolylamide; trans-4-glycylamino-D-prolyl-trans-4-((R)-2-hydroxy-4-phenylbutyrylamino)-L-proline 3-quinolylamide; trans-4-(N-methylglycylamino)-L-prolyl-trans-4-((R)-2-hydroxy-4-phenylbutyrylamino)-L-proline 3-quinolylamide; trans-4-((S)-3-amino-2-hydroxypropionylamino)-L-prolyl-trans-4-((R)-2-hydroxy-4-phenylbutyrylamino)-L-proline-3-quinolylamide; trans-4-aminomethyl-L-prolyl-trans-4-((R)-2-hydroxy-4-phenylbutyrylamino)-L-proline 3-quinolylamide; 4-(2-aminoethyl)-L-prolyl-trans-4-((R)-2-hydroxy-4-phenylbutyrylamino)-L-proline 3-quinolylamide; 1-(N-methylglycyl)-trans-4-amino-L-prolyl-trans-4-((R)-2-hydroxy-4-phenylbutyrylamino)-L-proline 3-quinolylamide; trans-4-amino-L-pipecolinoyl-trans-4-((R)-2-hydroxy-4-phenylbutyrylamino)-L-proline 3-quinolylamide; cis-4-amino-L-pipecolinoyl-trans-4-((R)-2-hydroxy-4-phenylbutyrylamino)-L-proline 3-quinolylamide; trans-4-glycylamino-L-pipecolinoyl-trans-4-((R)-2-hydroxy-4-phenylbutyrylamino)-L-proline 3-quinolylamide; cis-4-glycylamino-L-pipecolinoyol-trans-4-((R)-2-hydroxy-4-phenylbutyrylamino)-L-proline 3-quinolylamide; D-ornithyl-trans-4-(4-phenylbutanoyl)amino-L-proline-5-indanylamide; L-ornithyl-cis-4-(4-phenylbutanoyl)amino-L-proline-5-indanylamide; D-ornithyl-cis-4-(4-phenylbutanoyl)amino-L-proline 5-indanylamide; 4-hydroxy-L-ornithyl-trans-4-(4-phenylbutanoyl)amino-L-proline 5-indanylamide; trans-4-glycylamino-L-prolyl-D-homophenylalanine 3-quinolylamide; trans-4-amino-L-prolyl-D-homophenylalanine 3-quinolylamide; trans-4-glycylamino-L-prolyl-D-homophenylalanine 5-indanylamide; trans-4-glycylamino-L-prolyl-D-homophenylalanine 3,4-dimethylphenylamide; trans-4-glycylamino-L-prolyl-D-homophenylalanine 3,5-dimethylphenylamide; trans-4-glycylamino-L-prolyl-D-homophenylalanine 4-chloro-3-methylphenylamide; trans-4-glycylamino-L-prolylglycine 4-benzylphenylamide; trans-4-glyclamino-L-proline 4-phenoxyphenylamide; trans-4-glycylamino-L-proline 4-(4′-methylphenoxy)phenylamide; trans-4-glycylamino-L-proline 4-(4′-chlorophenoxy)phenylamide; trans-4-glycylamino-L-proline 4-phenylaminophenylamide; trans-4-glycylamino-L-proline 3-biphenylamide; trans-4-glycylamino-D-proline 3-biphenylamide; trans-4-glycylamino-L-proline 4-benzylphenylamide; trans-4-glycylamino-L-proline 4-tert-butylphenylamide; trans-4-glycylamino-L-proline 4-phenylbenzylamide; trans-4-glycylamino-L-proline 4-benzyloxyphenylamide; trans-4-glycylamino-L-proline 3-benzyloxyphenylamide; trans-4-glycylamino-L-proline 4-(phenylthiomethyl)phenylamide; trans-4-glycylamino-L-proline 4-benzylthiophenylamide; trans-4-((S)-3-amino-2-hydoxypropionylamino)-L-proline 4-phenoxyphenylamide; trans-4-(2-aminoethylsulfonylamino)-L-proline 4-phenoxyphenylamide; trans-4-glycylamino-L-proline 4-phenylthiazol-2-ylamide; trans-4-glycylamino-L-proline 3-(6-benzyl)quinolylamide; trans-4-amino-L-pipecolinoyl-(4-phenoxyphenyl)amide; trans-4-glycylamino-L-pipecolinoyl 4-phenoxyphenylamide; trans-4-aminomethyl-L-proline 4-phenoxyphenylamide; 1-(trans-4-glycylamino-L-prolyl)-4-(3-chlorophenyl)piperazine; 1-[trans-4-((2S)-3-amino-2-hydroxypropionylamino)-D-prolyl]-4-(3-chloro-2-methylphenyl)piperazine; 1-(N-trans-4-glycylamino-L-prolyl)-4-(4-chlorophenyl)piperazine; 1-(trans-4-glycylamino-L-prolyl)-4-(2-chlorophenyl)piperazine; 1-(trans-4-aminomethyl-L-prolyl)-4-(3-chloro-2-methylphenyl)piperazine; 1-(trans-4-glycylamino-L-prolyl)-4-(4-phenylbutanoyl)piperazine; (2R)-4-benzyl-1-(trans-4-glycylamino-D-prolyl)-2-phenethylpiperazine; 1-(trans-4-glycylamino-L-prolyl)-4-(4-benzyloxyphenoxy)piperidine; 1-(trans-4-glycylamino-L-prolyl)-4-(3,5-dichlorophenoxy)piperidine; 1-(trans-4-glycylamino-D-prolyl)-4-(3,5-dichlorophenoxy)piperidine; trans-4-glycylamino-L-prolyl-4-(2-chloro-5-methylphenoxy)piperidine; (2S,4R)-4-glycylamino-2-(4-biphenyloxy)methylpyrrolidine; (2S,4R)-4-glycylamino-2-(3-biphenyloxy)methylpyrrolidine; (2R,4S)-4-glycylamino-2-(4-biphenyloxy)methylpyrrolidine; (2R,4S)-4-glycylamino-2-(3-biphenyloxy)methylpyrrolidine, trans-4-(3-biphenyloxy)-L-proline 2-aminoethylamide; (2S,4R)-2-(2-amino-1-hydroxyethyl)-4-(3-biphenyloxy)pyrrolidine; 1-(N-trans-4-glycylamino-L-prolylamino)-3-(4-phenylpropanoylamino)benzene; 2-(trans-4-glycylanino-L-prolylamino)-6-(4-phenylpropanoylamino)pyridine; (2S,4R)-4-glycylamino-2-((E and Z)-4-phenylstyryl)pyrrolidine; globomycin (glycine, N-(N-(N-(N-(N-(3-hydroxy-2-methyl-1-oxononyl)-N-methylleucyl)-L-alloisoleucyl)-L-seryl)-L-allothreonyly, rho-lactone); carbonyl cyanide m-chlorophenylhydrazone (CCCP); pyridoquinolone; MC-04,124 ((2R,4R)-4-(aminomethyl)-N-[(2R)-1-oxo-4-phenyl-1-(quinolin-6-ylamino)butan-2-yl]pyrrolidine-2-carboxamide); or MC-02,595 (D-ornithine-D-homophenylalanine-3-aminoquinoline).

Contrary to the disclosure in WO 2016/080846, the inventors have found that halogenated salicylanilides, such as niclosamide, oxyclozanide, rafoxanide, and closantel, are effective against N. gonococcus without the need for a baterial efflux inhibitor. This simplifies the treatment and minimises patient exposure to additonal agents such as bacterial efflux pump inhibitor. Accordingly, the halogenated salicylanilide, such as niclosamide, oxyclozanide, rafoxanide, or closantel, may be used in the absence of concurrent administration with a bacterial efflux inhibitor, in particular any of the bacterial efflux inhibitors disclosed in WO 2016/080846, in the uses, methods, compositions and devices as disclosed herein.

Agents that Increase the Permeability of a Bacterial Cell Membrane

WO 2017/200396 refers to polymyxins, such as polymyxin B, structural/functional analogues of polymyxin B, polymyxin E (i.e. colistin), structural/functional analogues of polymyxin E, and cationic or anionic peptides that disrupt cell membrane homeostasis and/or polarity, e.g. gramicidin, as examples of agents that increase the permeability of the bacterial cell membrane.

Further examples of agents that in WO 2017/200396 are asserted to increase the permeability of bacterial cell membranes are hyperosmotic solutions, calcium ion chelators, surfactants, and/or polarity and receptor mediated permeabilizing agents including drug based agents that increase permeability of a bacterial cell membrane, as well as combinations thereof.

Examples of calcium ion chelators mentioned in WO 2017/200396 are iminodiacetic acid (IDA), nitriloacetic acid (NTA), ethylenediaminomonoacetic acid (EDMA), ethylenediaminodiacetic acid (EDDA), and ethylenediaminotetraacetic acid (EDTA).

Examples of ionic surfactants mentioned in WO 2017/200396 are sodium taurodihydrofusidate, sodium salicylate, sodium caprate, and sodium glycocholate.

Examples of non-ionic surfactants mentioned in WO 2017/200396 are cholylsarcosine, isopropyl myristate, partially hydrolyzed triglycerides, fatty-acid sugar derivatives, and oleic acid derivatives.

Reference is in WO 2017/200396 made to Hurdle et al. (2011) and Guihelemelli et al. (2013) for examples of cationic or anionic peptides that disrupt cell membrane homeostasis and/or polarity.

Examples of drug based agents that increase permeability of a bacterial cell membrane mentioned in WO 2017/200396 are polymyxins. Structural/functional analogues of polymyxin B, which differ in the N-terminal fatty acyl group and amino acid residue at position-6 and position-7 are mentioned. Structural/functional analogues of polymyxin E, which differ in the N-terminal fatty acyl group and amino acid residue at position-6 and position-7 are mentioned.

Contrary to the disclosure in WO 2017/200396, the inventors have found that halogenated salicylanilides, such as niclosamide, oxyclozanide, rafoxanide, and closantel, are effective against N. gonococcus in the absence of concurrent administration of an agent selected from the group consisting of polymyxins and gramicidin. Accordingly, the halogenated salicylanilide, such as niclosamide, oxyclozanide, rafoxanide, or closantel, may be used in the absence of concurrent administration with an agent that increases the permeability of a bacterial cell membrane, in particular any of the permeability enhancers disclosed in WO 2017/200396, in the uses, methods, compositions and devices as disclosed herein.

### Pharmaceutical Compositions

The halogenated salicylanilide may be administered to the subject in the form of a pharmaceutical composition, such as a topical formulation, comprising the halogenated salicylanilide, or a pharmaceutically acceptable salt or solvate thereof, and a pharmaceutically acceptable excipient.

Conventional procedures for the selection and preparation of suitable pharmaceutical formulations are described in, for example, “Pharmaceuticals—The Science of Dosage Form Designs”, M. E. Aulton, Churchill Livingstone, 1988.

The composition may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intraperitoneal dosing or as a suppository for rectal dosing). Suitably the composition is in a form suitable for oral administration.

The compositions may be obtained by conventional procedures using conventional pharmaceutical excipients, well known in the art. Thus, compositions intended for oral use may contain, for example, one or more colouring, sweetening, flavouring and/or preservative agents.

### Dosage

The amount of active ingredient that is combined with one or more excipients to produce a single dosage form will necessarily vary depending upon the subject treated and the particular route of administration.

For example, a formulation in a unit dose form such as a tablet or capsule intended for oral administration or another dose form to be administered parenterally to humans will generally contain, for example, from 0.1 mg to 5 mg, for example from 0.5 mg to 5 g, from 0.5 to 2 000 mg or from 10 to 500 mg of the halogenated salicylanilide, such as niclosamide, oxyclozanide, rafoxanide, or closantel, or a pharmaceutically acceptable salt or solvate thereof, compounded with an appropriate and convenient amount of excipients which may vary from about 5 to about 98 percent by weight of the total composition.

For example, a formulation intended for topical administration to humans, including adults, children and infants, will generally contain, for example, within the range of from 0.01 to 20 percent by weight of the total composition (i.e. % w/w), for example within the range of from 0.5 to 5% w/w, within the range of from 2 to 5% w/w or within the range of from 5 to 15% w/w, of the halogenated salicylanilide, such as niclosamide, oxyclozanide, rafoxanide, or closantel, or a pharmaceutically acceptable salt or solvate thereof, compounded with an appropriate and convenient amount of excipients which may vary, for example, within the range of from about 80 to about 99.99 percent by weight (i.e. % w/w) of the total composition.

The size of the dose of the halogenated salicylanilide, such as oxyclozanide, niclosamide, rafoxanide, or closantel, or a pharmaceutically acceptable salt or solvate thereof, for the treatment of the N. gonococcus infections described herein will naturally vary according to the nature and severity of the conditions, the age and sex of the animal or patient and the route of administration, according to well-known principles of medicine.

The halogenated salicylanilide, such as oxyclozanide, niclosamide, rafoxanide, or closantel, or a pharmaceutically acceptable salt or solvate thereof, will generally be administered in a dose of about 0.001 to about 75 mg/kg, for example from about 0.013 to about 66.7 mg/kg, about 0.5 to about 30 mg/kg or from about 2.5 to about 30 mg/kg. The halogenated salicylanilide, such as oxyclozanide, niclosamide, rafoxanide, or closantel, or a pharmaceutically acceptable salt or solvate thereof, may be administered within these dosage ranges to the subject from 1 to 4 times per day. The dosage may be administered by any suitable route, for example parenterally, orally or topically. A particular route of administration which is generally applicable to all of the uses of the halogenated salicylanilides described herein is the oral administration of the halogenated salicylanilide, such as oxyclozanide, niclosamide, rafoxanide, or closantel, or a pharmaceutically acceptable salt or solvate thereof, to the subject.

The particular dosage regimen used to treat a subject will depend on a number of factors that may readily be determined, such as the severity of the condition and its responsiveness to initial treatment, but will normally involve one or more administrations per day on an ongoing basis. The effective dosage of the pharmaceutical composition of the present invention varies from the formulation, administration pathway, age, weight and gender of a human or animal or with a disease caused by N. gonococcus species.

### Therapeutic Use

As described hereinbefore the halogenated salicylanilide, such as oxyclozanide, niclosamide, rafoxanide, or closantel, or a pharmaceutically acceptable salt or solvate thereof, is used for the treatment of an infection caused by N. gonococcus bacteria. The halogenated salicylanilide, such as oxyclozanide, niclosamide, rafoxanide, or closantel, or a pharmaceutically acceptable salt or solvate thereof, may act to kill or eradicate the infection from the subject, thus providing a bactericidal effect. Alternatively, the halogenated salicylanilide, such as oxyclozanide, niclosamide, rafoxanide, or closantel, or a pharmaceutically acceptable salt or solvate thereof, may inhibit growth or replication of the bacteria thus producing a bacteriostatic effect. In the context of the present invention, treatment of a condition encompasses both therapeutic and prophylactic treatment, of either an infectious or a non-infectious condition, in a subject for example a mammal such as a human or animal, but in particular a human. It may involve complete or partial eradication of the condition, removal or amelioration of associated symptoms, arresting subsequent development of the condition, and/or prevention of, or reduction of risk of, subsequent occurrence of the condition.

Generally, the halogenated salicylanilide, such as oxyclozanide, niclosamide, rafoxanide, or closantel, or a pharmaceutically acceptable salt or solvate thereof, will be administered to a subject experiencing symptoms of a N. gonococcus infection.

In an alternative embodiment, the halogenated salicylanilide, such as oxyclozanide, niclosamide, rafoxanide, or closantel, or a pharmaceutically acceptable salt or solvate thereof, is for use in the treatment of a N. gonococcus in a subject, wherein the subject is asymptomatic. Such uses may be useful to eradicate or inhibit a N. gonococcus infection in a subject that is at risk of developing N. gonococcus associated infectious disease.

In embodiments, the halogenated salicylanilide, such as oxyclozanide, niclosamide, rafoxanide, or closantel, or a pharmaceutically acceptable salt or solvate thereof, is for use in the treatment of a N. gonococcus infection selected from (i) a urogenital, an anorectal, a pharyngeal and/or a conjunctival gonococcal infection, or (ii) a disseminated gonococcal infection.

In embodiments, the infectious disease caused by Neisseria gonococcus is selected from adult gonococcal conjunctivitis or neonatal gonococcal conjunctivitis, in particular neonatal gonococcal conjunctivitis.

In embodiments, the infectious disease caused by Neisseria gonococcus is gonococcal conjunctivitis, and the halogenated salicylanilide, such as oxyclozanide, niclosamide, rafoxanide, or closantel, or a pharmaceutically acceptable salt or solvate thereof, is topically administered to the subject.

In embodiments, the infectious disease caused by Neisseria gonococcus is neonatal gonococcal conjunctivitis, and the halogenated salicylanilide, such as oxyclozanide, niclosamide, rafoxanide, or closantel, or a pharmaceutically acceptable salt or solvate thereof, is topically administered to the subject's eye (infant's eye) to treat a conjunctival gonococcal infection.

In embodiments, the infectious disease caused by Neisseria gonococcus is neonatal gonococcal conjunctivitis, and the halogenated salicylanilide, such as oxyclozanide, niclosamide, rafoxanide, or closantel, or a pharmaceutically acceptable salt or solvate thereof, is topically administered to the subject's eye (infant's eye) to prevent a conjunctival gonococcal infection.

In embodiments, the infectious disease caused by Neisseria gonococcus is urogenital gonorrhoea, such as asymptomatic or symptomatic urogenital gonorrhoea.

In embodiments, the infectious disease caused by Neisseria gonococcus is urogenital gonorrhoea, such as asymptomatic or symptomatic urogenital gonorrhoea, and the halogenated salicylanilide, such as oxyclozanide, niclosamide, rafoxanide, or closantel, or a pharmaceutically acceptable salt or solvate thereof, is topically administered to the subject's urogenital tract to treat a urogenital gonococcal infection.

In embodiments, the infectious disease caused by Neisseria gonococcus is urogenital gonorrhoea, such as asymptomatic or symptomatic urogenital gonorrhoea, and the halogenated salicylanilide, such as oxyclozanide, niclosamide, rafoxanide, or closantel, or a pharmaceutically acceptable salt or solvate thereof, is topically administered to the subject's urogenital tract to prevent a urogenital gonococcal infection.

In embodiments, the infectious disease caused by Neisseria gonococcus is anorectal gonorrhoea, such as asymptomatic or symptomatic anorectal gonorrhoea.

In embodiments, the infectious disease caused by Neisseria gonococcus is anorectal gonorrhoea, such as asymptomatic or symptomatic anorectal gonorrhoea, and the halogenated salicylanilide, such as oxyclozanide, niclosamide, rafoxanide, or closantel, or a pharmaceutically acceptable salt or solvate thereof, is topically administered to the subject's anorectal tract to treat an anorectal gonococcal infection.

In embodiments, the infectious disease caused by Neisseria gonococcus is anorectal gonorrhoea, such as asymptomatic or symptomatic anorectal gonorrhoea, and the halogenated salicylanilide, such as oxyclozanide, niclosamide, rafoxanide, or closantel, or a pharmaceutically acceptable salt or solvate thereof, is topically administered to the subject's anorectal tract to prevent an anorectal gonococcal infection.

In one embodiment, the halogenated salicylanilide, such as oxyclozanide, niclosamide, rafoxanide, or closantel, or a pharmaceutically acceptable salt or solvate thereof, is administered (via any available route providing the desired systemic or local action) to the subject concurrently with another therapeutic agent, provided said other therapeutic agent is not a bacterial efflux pump inhibitor and provided said other therapeutical agent is not an agent selected from the group consisting of polymyxins and gramicidin, in any of the treatments of the N. gonococcus infections infections described herein.

The halogenated salicylanilide, such as oxyclozanide, niclosamide, rafoxanide, or closantel, or a pharmaceutically acceptable salt or solvate thereof, may be administered (via any available route providing the desired systemic or local action) to the subject concurrently with another antibiotic being used to treat another infection in the subject, provided the antibiotic is not a polymyxin or a gramicidin. For example, it may be that the halogenated salicylanilide, such as oxyclozanide, niclosamide, rafoxanide, or closantel, or a pharmaceutically acceptable salt or solvate thereof, is administered to the subject concurrently with an antibiotic being used to treat an infection other than a N. gonococcus infection. The antibiotic used to treat the other infection may, for example, be one or more antibiotics selected from a penicillin, a cephalosporin, a carbapenem, a monobactam (for example a β-lactam antibiotic), a fusidane, a fluoroquinolone, a tetracycline, a glycylcycline, phenicol (for example chloramphenicol), a macrolide, a macrocyclic (for example fidaxomicin), a rifamycin, a ketolide, a lincosamide, an oxazolidinone, an aminocyclitol, a glycopeptide, an aminoglycoside, a lipopeptide, an antimycobacterial, a nitromidazole, bacitracin, mupiricin, a pleuromutilin, a rifamycin, a sulphonamide and trimethoprim, or a combination of two or more thereof. More preferably, the halogenated salicylanilide is administered to the subject in the absence of any other antibiotic agent.

In embodiments, the halogenated salicylanilide, such as oxyclozanide, niclosamide, rafoxanide, or closantel, or a pharmaceutically acceptable salt or solvate thereof, is administered (via any available route providing desired systemic or local action) to the subject concurrently with another therapeutic agent which is an antibiotic active against Chlamydia trachomatis. An example of such an antibiotic is azithromycin. Azithromycin may be administered as an oral tablet, an oral capsule, an oral suspension, an intravenous injection, granules in sachet for suspension, and ophthalmic solution.

In embodiments, the halogenated salicylanilide, such as oxyclozanide, niclosamide, rafoxanide, or closantel, or a pharmaceutically acceptable salt or solvate thereof, is administered to the subject concurrently with another antibiotic active against a N. gonococcus infection other than the halogenated salicylanilide itself, provided the antibiotic is not a polymyxin or a gramicidin.

In embodiments, the infectious disease caused by Neisseria gonococcus is gonococcal conjunctivitis and the halogenated salicylanilide, such as oxyclozanide, niclosamide, rafoxanide, or closantel, or a pharmaceutically acceptable salt or solvate thereof, is topically administered to the subject's eye to treat the conjunctival gonococcal infection and another antibiotic active against a N. gonococcus infection other than the halogenated salicylanilide itself, provided the antibiotic is not a polymyxin or a gramicidin, is concurrently administered (e.g. orally or topically) to the subject.

In embodiments, the infectious disease caused by Neisseria gonococcus is neonatal gonococcal conjunctivitis and the halogenated salicylanilide, such as oxyclozanide, niclosamide, rafoxanide, or closantel, or a pharmaceutically acceptable salt or solvate thereof, is topically administered to the infant's eye to treat, including prevent, the conjunctival gonococcal infection and another antibiotic active against a N. gonococcus infection other than the halogenated salicylanilide itself, provided the antibiotic is not a polymyxin or a gramicidin, is concurrently administered (e.g. orally or topically) to the infant.

In embodiments, the infectious disease caused by Neisseria gonococcus is urogenital gonorrhoea, such as asymptomatic or symptomatic urogenital gonorrhoea, and the halogenated salicylanilide, such as oxyclozanide, niclosamide, rafoxanide, or closantel, or a pharmaceutically acceptable salt or solvate thereof, is topically administered to the subject's urogenital tract to treat the urogenital gonococcal infection and another antibiotic active against a N. gonococcus infection other than the halogenated salicylanilide itself, provided the antibiotic is not a polymyxin or a gramicidin, is concurrently administered (e.g. orally or topically) to the subject.

In embodiments, the infectious disease caused by Neisseria gonococcus is anorectal gonorrhoea, such as asymptomatic or symptomatic anorectal gonorrhoea, and the halogenated salicylanilide, such as oxyclozanide, niclosamide, rafoxanide, or closantel, or a pharmaceutically acceptable salt or solvate thereof, is topically administered to the subject's anorectal tract to treat the anorectal gonococcal infection and another antibiotic active against a N. gonococcus infection other than the halogenated salicylanilide itself, provided the antibiotic is not a polymyxin or a gramicidin, is concurrently administered (e.g. orally or topically) to the subject.

In embodiments, the halogenated salicylanilide, such as oxyclozanide, niclosamide, rafoxanide, or closantel, or a pharmaceutically acceptable salt or solvate thereof, is administered to the subject concurrently with another antibiotic selected from a cephalosporin, a tetracycline or a combination thereof.

In embodiments, the halogenated salicylanilide, such as oxyclozanide, niclosamide, rafoxanide, or closantel, or a pharmaceutically acceptable salt or solvate thereof, is administered to the subject concurrently with another antibiotic selected from cephalexin, cefixime, ceftriaxone, doxycycline, azithromycin or any combinations thereof.

In embodiments, the halogenated salicylanilide, such as oxyclozanide, niclosamide, rafoxanide, or closantel, or a pharmaceutically acceptable salt or solvate thereof, is administered to the subject in the absence of any concurrent administration of another antibiotic agent.

In embodiments, the halogenated salicylanilide, such as oxyclozanide, niclosamide, rafoxanide, or closantel, or a pharmaceutically acceptable salt or solvate thereof, is administered to the subject in the absence of any concurrent administration of an agent that increases the permeability of the bacterial cell membrane, such as polymyxins, gramicidins or any structural/functional analogues thereof.

In embodiments, the halogenated salicylanilide, such as oxyclozanide, niclosamide, rafoxanide, or closantel, or a pharmaceutically acceptable salt or solvate thereof, is administered to the subject in the absence of any concurrent administration of a bacterial efflux pump inhibitor, such as phenylalanine-arginine β-napthylamide or 2,3-dibromomaleimide.

In embodiments, the halogenated salicylanilide, such as oxyclozanide, niclosamide, rafoxanide, or closantel, or a pharmaceutically acceptable salt or solvate thereof, is administered to the subject in the absence of any concurrent administration of a commensal species of Neisseria, such as Neisseria elongate and Neisseria polysaccharea, or a portion or extract thereof, in particular any extracts of commensal species of Neisseria as disclosed in WO 2016/036839 A1.

In embodiments, the halogenated salicylanilide, such as oxyclozanide, niclosamide, rafoxanide, or closantel, or a pharmaceutically acceptable salt or solvate thereof, is administered to the subject in the absence of any concurrent administration of (i) an agent that increase the permeability of the bacterial cell membrane, (ii) a bacterial efflux pump inhibitor, and/or (iii) a commensal species of Neisseria, such as Neisseria elongate and Neisseria polysaccharea, or a portion or extract thereof.

Reference to administration “concurrently” herein includes the separate, simultaneous or separate administration of the halogenated salicylanilide with the other therapy. The halogenated salicylanilide may be administered to the subject administered by the same or different routes of administration, for example oral, intravenously, subcutaneously, or topically). The halogenated salicylanilide and the other therapy may be administered as a combined preparation; however, generally they will be administered as separate dosage forms to enable the dose and dosing regimen of each to be tailored accordingly.

The presence of a N. gonococcus infection in a subject may be diagnosed using conventional methods, for example infection may be suspected from subject exhibiting symptoms of a N. gonococcus associated infectious disease. Infection may also be diagnosed using known methods for example by taking a sample of fluid from the symptomatic area (e.g. penis, vagina, rectum, throat, eye). If a joint or blood infection is suspected, a blood sample or fluid from the symptomatic joint is taken. The sample is then cultured on selective media.

N. gonococcus bacteria are expected to exhibit a low frequency of spontaneous mutation in the presence of the halogenated salicylanilides described herein. Therefore, it is expected that the risk of resistance to the halogenated salicylanilide emerging will be low.

### Subjects

The subject topically treated with the halogenated salicylanilide may be a warm-blooded animal, preferably the subject is a human. Also contemplated is the treatment of a non-human subject such as a warm blooded non-human mammal. For example, the subject may be a commercial animal such as livestock (e.g. cows, sheep, chickens, pigs, geese, ducks, goats, etc.). In other embodiments subject is a companion animal such as a cat, dog or horse. In some embodiments, the subject is a dog or a cat. In a particular embodiment, the subject is a dog.

### EXAMPLES

In the examples below, the effect of niclosamide is compared to the effect of other antibiotics on various Gram-negative bacteria bacteria and the effect of niclosamide, closantel, rafoxanide and oxyclozanide on N. gonococcus is shown.

### Example 1: MIC Determinations

### Materials

Niclosamide (anhydrous) was stored at −20° C. as a powder prior to testing.

Stock solutions of the reference compounds tested were prepared using DMSO. Additional dilutions were made from the stock solutions to achieve a broader testing range for some groups of isolates. All compounds were in solution prior to use.

Information regarding compound source, catalog and lot numbers, and testing ranges for niclosamide and the reference compounds are detailed in Table 1.

TABLE 1

| | | | | Testing |
|---|---|---|---|---|
| | | | | ranges |
| Compound | Supplier | Catalog No. | Lot No. | [μg/ml] |
| Niclosamide | AntibioTx | — | 143001W01C | 0.008-8 |
| Levofloxacin | Sigma | 28266 | BCBF7004V | 0.004-32 |
| Cephalexin | Sigma | C4895 | 074K0635 | 0.03-32 |
| Doxycycline | Sigma | D9891 | BCBF9827V | 0.008-8 |
| Amikacin | Sigma | A2324 | 058K0803 | 0.06-64 |
| Meropenem | USP | 1392454 | J0K434 | 0.004-4 |

Appropriate quality control strains were included on each day of the assay to confirm the accuracy of the drug dilutions and reproducibility of the results. The Micromyx isolate number and corresponding ATCC number are shown in Table 2.

TABLE 2

| | Micromyx | |
|---|---|---|
| Bacteria | isolate number | ATCC No. |
| Escherichia coli | MMX 102 | ATCC 25922 |
| (E. coli) | | |
| Pseudomonas aeruginosa | MMX 103 | ATCC 27853 |
| (P. aeruginosa) | | |
| Neisseria gonococcus/ | MMX 683 | ATCC 49226 |
| Neisseria gonorrhoeae | | |
| (N. gonococcus/N. | | |
| gonorrhoeae) | | |
| Enterobacter cloacae | MMX 6089 | |
| (E. cloacae) | | |
| Klebsiella pneumoniae | MMX 214 | ATCC 13883 |
| (K. pneumoniae) | | |
| Acinetobacter baumannii | MMX 1630 | ATCC 19606 |
| (A. baumannii) | | |
| Stenotrophomonas | MMX 4746 | |
| maltophila | | |
| (S. maltophila) | | |

All isolates were maintained at −80° C. Prior to testing, the isolates were streaked from the frozen vials onto the appropriate agar media: for aerobes, Tryptic Soy Agar (TSA) +5% Laked Sheep Blood, Remel, catalog no. R01202; lot no. 212574; for Neisseria, Chocolate Agar (CA), Becton Dickinson (BD), catalog no. 221267, lot no. 6349730. Aerobes were incubated overnight at 35° C. Neisseria plates were incubated in a CO₂ incubator overnight at 35° C.

Broth microdilution assay: Mueller Hinton II broth (MHB II, BBL, BD, catalog no. 212322; lot no. 5257869) was used for testing most aerobes.

Agar dilution MIC assay: Neisseria gonococcus (Neisseria gonorrhoeae) was tested on Difco GC medium Base (BD, catalog no. 228950; lot no. 4274618) supplemented with 1% IsoVitaleX Enrichment (BD, catalog no. 211876; lot no. 7101929).

Broth Microdilution MIC Testing

MIC values were determined using a broth microdilution procedure described by CLSI, see reference (1). Automated liquid handlers (Multidrop 384, Labsystems, Helsinki, Finland; Biomek 2000 and Biomek FX, Beckman Coulter, Fullerton Calif.) were used to conduct serial dilutions and liquid transfers.

The wells in Columns 2 through 12 of two standard 96-well microdilution plates (Costar 3795, Corning Inc., Corning, N.Y.) were filled with 150 μL of diluent specific to the compound tested. Three hundred microliters of each comparator and investigational drug solution (40× or 100×) was added to one well in Column 1 of the plates. The Biomek 2000 was used to complete the serial transfers through Column 11. The wells of Column 12 contained no drug and were the organism growth control wells. These panels were the “mother plates.”

Because drugs were prepared at either 40× or 100×, the “daughter plates” were loaded with 185-188 μL of the appropriate test media for the tested organism, using the Multidrop 384 or by hand. The daughter plates were prepared on the Biomek FX instrument, which transferred 2-5 μL of drug solution from each well of a mother plate to the corresponding well of each daughter plate in a single step. The wells of the daughter plates ultimately contained 185-188 μL of broth appropriate to the organism, 2-5 μL of drug solution, and 10 μL of bacterial inoculum prepared in broth.

Standardized inoculum of each organism was prepared per CLSI methods, see reference (1). The inoculum for each organism was dispensed into sterile reservoirs divided by length (Beckman Coulter), and the Biomek 2000 was used to inoculate the plates. Daughter plates were placed on the Biomek 2000 work surface in reverse orientation so that inoculation took place from low to high drug concentration. The Biomek 2000 delivered 10 μL of standardized inoculum into each well. This yielded a final cell density in the daughter plates of approximately 5×10⁵ CFU/mL.

Plates were stacked 3 high, covered with a lid on the top plate, placed in plastic bags, and incubated at 35° C. for approximately 18 to 20 hr. Following incubation, the microplates were removed from the incubator and viewed from the bottom using a plate viewer. For the test article and each comparator, an un-inoculated solubility control plate was observed for evidence of drug precipitation. The MIC was read and recorded as the lowest concentration of drug that inhibited visible growth of the organism.

### Agar Dilution MIC Methodology

Neisseria gonococcus (N. gonorrhoeae) were assayed using a reference agar dilution method, see reference (2). Drug dilutions and drug-supplemented agar plates were prepared manually. Neisseria gonococcus was grown on Chocolate agar in a CO₂ incubator overnight at 35° C. prior to assay. Neisseria gonococcus was grown in a CO₂ incubator overnight at 35° C. The MIC was read per CLSI guidelines, see reference (3).

### References

(1) Clinical Laboratory and Standards Institute (CLSI). Methods for Dilution Antimicrobial Susceptibility Tests for Bacteria That Grow Aerobically; Approved Standard—Tenth Edition. CLSI document M07-A10 [ISBN 1-56238-988-2]. CLSI, 950 West Valley Road, Suite 2500, Wayne, Pa. 19087 USA, 2015.
(2) CLSI. Methods for Antimicrobial Susceptibility Testing of Anaerobic Bacteria; Approved Standard—Seventh Edition. CLSI document M11-A8. Clinical and Laboratory Standards Institute, 940 West Valley Road, Suite 1400, Wayne, Pa. 19087-1898 USA, 2012.
(3) CLSI. Performance Standards for Antimicrobial Susceptibility Testing. 27th ed. CLSI supplement M100-27. Clinical and Laboratory Standards Institute, 950 West Valley Road, Suite 2500, Wayne, Pa. 19087 USA, 2017.

The results of evaluating niclosamide against N. gonorrhoeae are shown in Table 4 and against the other Gram-negative pathogens are shown in Table 3.

Results against Gram-negative pathogens (Table 3)

Two E. coli strains were evaluated in this study. Against these strains, niclosamide had MIC values of >8 μg/mL, including the ΔacrAB efflux pump strain. Meropenem and levofloxacin were the most active comparators, with MIC values of 0.015 μg/mL.

Against the single K. pneumoniae isolate, niclosamide had an MIC value of >8 μg/mL; levofloxacin and meropenem were the most active comparators in this study, with MIC values of 0.03 μg/mL.

Against the E. cloacae isolate tested, niclosamide had an MIC value of >8 μg/mL; levofloxacin was the most active comparator in this study, with MIC value of 0.03 μg/.

Against P. aeruginosa ATCC 27853/MMX 103, niclosamide had an MIC value of >8 μg/mL; meropenem was the most active comparator, with an MIC value of 0.25 μg/mL.

Against the S. maltophila isolate tested, niclosamide had an MIC value of >8 μg/mL; levofloxacin and doxycycline were the comparators with the best activity (0.5 μg/mL).

Against the A. baumannii isolate tested, niclosamide had an MIC value of >8 μg/mL; levofloxacin and doxycycline had the best activity against this organism, with MIC values of 0.5 μg/mL for each.

MIC values for levofloxacin, ceftazidime, doxycycline, amikacin, meropenem, and trimethoprim-sulfamethoxazole against E. coli ATCC25922/MMX 102; and for levofloxacin, ceftazidime, amikacin, meropenem, and trimethoprim-sulfamethoxazole against P. aeruginosa ATCC 27853/MMX 103 were within published CLSI QC ranges, thus validating the assay.

Results Against N. gonococcus (N. gonorrhoeae) (Table 4)

Niclosamide was tested against N. gonorrhoeae ATCC49226/MMX 683; the MIC value was 0.12 μg/mL, whereas that of the comparator ciprofloxacin was 0.004 μg/mL. The MIC value for ciprofloxacin was within published CLSI QC ranges, thus validating the assay.

### Conclusion

In summary, niclosamide was evaluated against 7 Gram-negative bacterial pathogens for in vitro activity. Surprisingly, good in vitro activity was observed against N. gonococcus (N. gonorrhoeae) while the antibacterial effect of niclosamide on the other Gram-negative bacterial pathogens tested was found to be inadequate.

Niclosamide, or a pharmaceutically acceptable salt or solvate thereof, are therefore expected to provide a sufficient antibacterial effect on N. gonococcus to be useful as single-compound treatment of N. gonococcus infections.

TABLE 3
In vitro activity of nicloamide against Gram-negative pathogens
Minimal Inhibitory Concentration (μg/ml

| | Culture | | | | |
|---|---|---|---|---|---|
| | Number | Culture | | | |
| Organism | MMX/ATCC | Type | Niclosamide | Levofloxacin | Cephalexin |
| Escherichia coli | 10225922 | QC | >8 | 0.015 | 8 |
| | | | | (0.008 0.06)¹ | |
| Escherichia coli | 120 | acrAB efflux | >8 | ≤0.004 | 8 |
| | | pump | | | |
| | | knockout | | | |
| Klebsiella pneumoniae | 21413883 | Susceptible | >8 | 0.03 | 4 |
| Enterobacter cloacae | 6089 | Susceptible | >8 | 0.03 | >32 |
| Pseudomonas aeruginosa | 10327853 | Susceptible; | >8 | 1 | >32 |
| | | QC | | (0.5-4) | |
| Stenotrophomonas maltophila | 4746 | Susceptible | >8 | 0.5 | >32 |
| Acinetobacter baumanni | 163019606 | Susceptible | >8 | 0.5 | >32 |

| | | | | | Trimethoprim/ |
|---|---|---|---|---|---|
| Organism | Ceftazidime | Doxycycline | Amikacin | Meropenem | Sulfamethoxazole |
| Escherichia coli | 0.25 | 0.5 | 4 | 0.015 | 0.12/2.38 |
| | (0.06-0.5) | (0.5-2) | (0.5-4) | (0.008-0.06) | (≤0.5/9.5) |
| Escherichia coli | 0.12 | 0.25 | 0.5 | 0.015 | ≤0.015/0.3 |
| Klebsiella pneumoniae | 0.5 | 1 | 0.5 | 0.03 | 1/19 |
| Enterobacter cloacae | 8 | 1 | 1 | 0.06 | 0.5/9.5 |
| Pseudomonas aeruginosa | 1 | >8 | 2 | 0.25 | >16/304 |
| | (1-4) | | (1-4) | (0.12-1) | (8/152-32/606) |
| Stenotrophomonas maltophila | 4 | 0.5 | 2 | 2 | 0.12/2.38 |
| Acinetobacter baumanni | 4 | 0.5 | 16 | 1 | >16/304 |

¹CLSI QC range shown in parentheses
BL+ = beta-lactamase positive

TABLE 4
In vitro activity of niclosamide and ciprofloxacvin against Neisseria gonococcus (μg/ml)
Minimal Inhibitory Concentration (ng/rnl)

| Organism | MMX/ATCC | Phenotype | Niclosamide | Ciprofloxacin |
|---|---|---|---|---|
| Neisseria gonorrhoeae | 68349226 | QC | 0.12 | 0.004 |
| | | | | (0.001-0.008)¹ |

¹CLSI QC range shown in parentheses.

### Example 2

Neisseria gonorrhoeae (Neisseria gonocossus) were tested for antimicrobial susceptibility using agar dilution as described by the Clinical and Laboratory Standards Institute (CLSI), see reference (4).

### Materials

Stock solutions of niclosamide, oxyclosanide, rafoxanide and closantel (hereinafter referred to as antimicrobial agents) were prepared using DMSO. Stock solutions were freshly prepared and used on the same day or was stored at −70° C. for up to one month as single use aliquots when permissible.

The concentrations tested for each compound encompass the reported interpretive breakpoints and consist of 8 or more doubling dilutions, ensuring that there is one dilution below the lowest value of QC.

GC agar base +1% defined growth supplement.

Initial screening strains: Neisseria gonorrhoeae CDC panel consisting of 48 isolates. The strains were provided by Clinical and Environmental Microbiology Branch, Division of Healthcare Quality Promotion, CDC (Atlanta, US).

### Preparing Agar Dilution Plates

The following procedures were followed:

Dilution scheme for reference method: Prepare intermediate 100× antimicrobial agent solutions by making successive serial twofold dilutions. Then, add one part of the 100× antimicrobial solution to 9.9 parts of molten agar. Plates containing no antimicrobial agent solutions are made for use as growth controls. It is important that no longer than one hour elapses between the time that the stock solution is thawed, the dilutions are prepared and added to the base medium and the plates are poured, see reference (5).

### Procedure:

Add appropriate dilutions of antimicrobial solution to molten test agars that have been allowed to equilibrate in a water bath to 50° C.
Mix the agar and antimicrobial solution thoroughly and pour (20 mL) into Petri dishes on a level surface. Pour quickly after mixing to prevent cooling and partial solidification in the mixing container. Avoid generating bubbles when mixing.
Allow the agar to solidify at room temperature and store them in sealed plastic bags at 2 to 8° C. for up to five days. Preferably use with 24 hours.
Allow plates stored at 2 to 8° C. to equilibrate to room temperature before use. Make certain that the agar surface is dry before inoculating the plates. If necessary, place the plates in an incubator or laminar flow hood for approximately 30 minutes with their lids ajar to hasten drying of the agar surface.

Drug-free plates prepared from the base medium (with 1% defined growth supplement were used as growth controls.

### Preparing the Inoculum, See Reference (4)

The following procedures were followed:

Inoculum preparation: Prepare a standardized inoculum for the agar dilution method by suspending isolated colonies of Neisseria gonorrhoeae selected from an 18-24 hour-old chocolate agar plate directly in Mueller Hinton Broth (MHB) to achieve the density equivalent to a 0.5 McFarland standard and adjusted to obtain a cell density of ˜1-2×10⁸ CFU/mL. For N. gonorrhoeae, the McFarland solutions are made at a reading of ˜0.80 in densitometer (Grant Instruments DEN-1B Densitometer) because these values have been evaluated and correspond to the desired CFU/mL count.

Dilution of inoculum suspension: Cultures adjusted to the 0.5 McFarland standard contain approximately 1 to 2×10⁸ CFU/mL with most species, and the final inoculum required is 10⁴ CFU per spot. When using replicators with 3-mm pins that deliver ˜2.5 μL, dilute the 0.5 McFarland suspension 1:10 in sterile MHB to obtain a concentration of 10⁷ CFU/mL. The final inoculum on the agar will be approximately 10⁴ CFU per spot. Use the adjusted suspensions for final inoculation within 60 minutes of preparation, see reference (5).

### Inoculating Agar Dilution Plates

The following procedure was followed:
Arrange the tubes containing the adjusted and diluted bacterial suspensions (10⁷ CFU/mL) in order in a rack. Place an aliquot of each well-mixed suspension into the corresponding well in the replicator inoculum block (if being used).
Mark the agar plates for orientation of the inoculum spots.
Apply an aliquot of each inoculum (2.5 μL) to the agar surface, either with an inocula-replicating device or pipette, to obtain a final concentration of ˜10⁴ CFU/spot.
Inoculate a growth-control plate (no antimicrobial agent) first and then, starting with the lowest concentration, inoculate the plates containing the different antimicrobial concentrations. Inoculate a second growth control plate last to ensure isolate viability during the inoculation process.
Ten-fold serially dilute original inoculum suspension and plate samples at 10⁻⁶ and 10⁻¹ dilution to confirm cell density was within QC range (18 1-2×10⁸ CFU/mL).

### Incubating Agar Dilution Plates

The following procedure was followed: Allow the inoculated plates to stand at room temperature until the moisture in the inoculum spots has been absorbed into the agar, but no more than 30 minutes. Invert the plates and incubate at 36±1° C. in an atmosphere containing 5% CO₂ for 20 to 24 hours, see reference (4). All plates are placed in the incubator within 1 hour of strain suspension preparation.

### Determining Agar Dilution End Points

The following procedure was followed: Place the plates on a dark, nonreflecting surface to determine the end points. Record the MIC as the lowest concentration of antimicrobial agent that completely inhibits growth, disregarding a single colony or a faint haze caused by the inoculum. If two or more colonies persist in concentrations of the agent beyond an obvious end point, or if there is no growth at lower concentrations but growth at higher concentrations, check the culture purity and repeat the test if required.

### References

(4) CLSI. 2018. Performance standards for antimicrobial susceptibility testing. 28th ed. suppleent. CLSI document M100. Wayne, Pa.: Clinical and Laboratory Standards Institute; 2018.
(5) CDC. 2016. Gonococcal isolate surveillance program protocol, May 2016. https://www.cdc.qov/std/gisp/default.htm.
   Results Against N. gonorrhoea (Table 5)

Niclosamide was tested against 48 isolates of N. gonorrhoeae. MIC value was ≤50.25 μg/mL for each of the isolates.

Oxyclosanide was tested against 48 isolates of N. gonorrhoeae. MIC value was ≤50.25 μg/mL for each of the isolates.

Rafoxanide was tested against 48 isolates of N. gonorrhoeae. MIC value was ≤50.25 μg/mL for 46 of the isolates.

Closantel was tested against 48 isolates of N. gonorrhoeae. MIC value was ≤50.25 μg/mL for 44 of the isolates.

### Conclusion

Good in vitro activity of each of niclosamide, oxyclosanide, rafoxanide and closantel was observed against N. gonococcus (N. gonorrhoeae).

Each of niclosamide, oxyclosanide, rafoxanide and closantel, or pharmaceutically acceptable salts or solvates thereof, are therefore expected to provide a sufficient antibacterial effect on N. gonococcus to be useful as single-compound treatment of N. gonococcus infections.

TABLE 5
In vitro activity of niclosamide, oxyclozanide, rafoxanide and closantel
against Neisseria gonorrhoeae (Neisseria gonococcus)

| | Minimal inhibitory concentration [μg/ml] | | | | |
|---|---|---|---|---|---|
| Strain, | | | Oxyclo- | | |
| CDC # | Organism | Niclosamide | sanide | Rafoxanide | Closantel |
| 165 | Neisseria | ≤0.25 | ≤0.25 | ≤0.25 | 8 |
| | gonorrhoeae | | | | |
| 166 | Neisseria | ≤0.25 | ≤0.25 | ≤0.25 | ≤0.25 |
| | gonorrhoeae | | | | |
| 167 | Neisseria | ≤0.25 | ≤0.25 | ≤0.25 | ≤0.25 |
| | gonorrhoeae | | | | |
| 169 | Neisseria | ≤0.25 | ≤0.25 | ≤0.25 | ≤0.25 |
| | gonorrhoeae | | | | |
| 170 | Neisseria | ≤0.25 | ≤0.25 | ≤0.25 | ≤0.25 |
| | gonorrhoeae | | | | |
| 171 | Neisseria | ≤0.25 | ≤0.25 | ≤0.25 | ≤0.25 |
| | gonorrhoeae | | | | |
| 172 | Neisseria | ≤0.25 | ≤0.25 | ≤0.25 | ≤0.25 |
| | gonorrhoeae | | | | |
| 173 | Neisseria | ≤0.25 | ≤0.25 | ≤0.25 | 0.5 |
| | gonorrhoeae | | | | |
| 174 | Neisseria | ≤0.25 | ≤0.25 | ≤0.25 | ≤0.25 |
| | gonorrhoeae | | | | |
| 175 | Neisseria | ≤0.25 | ≤0.25 | ≤0.25 | ≤0.25 |
| | gonorrhoeae | | | | |
| 176 | Neisseria | ≤0.25 | ≤0.25 | ≤0.25 | ≤0.25 |
| | gonorrhoeae | | | | |
| 177 | Neisseria | ≤0.25 | ≤0.25 | ≤0.25 | 4 |
| | gonorrhoeae | | | | |
| 178 | Neisseria | ≤0.25 | ≤0.25 | ≤0.25 | ≤0.25 |
| | gonorrhoeae | | | | |
| 179 | Neisseria | ≤0.25 | ≤0.25 | ≤0.25 | ≤0.25 |
| | gonorrhoeae | | | | |
| 180 | Neisseria | ≤0.25 | ≤0.25 | ≤0.25 | ≤0.25 |
| | gonorrhoeae | | | | |
| 181 | Neisseria | ≤0.25 | ≤0.25 | ≤0.25 | ≤0.25 |
| | gonorrhoeae | | | | |
| 182 | Neisseria | ≤0.25 | ≤0.25 | 2 | ≤0.25 |
| | gonorrhoeae | | | | |
| 183 | Neisseria | ≤0.25 | ≤0.25 | ≤0.25 | ≤0.25 |
| | gonorrhoeae | | | | |
| 184 | Neisseria | ≤0.25 | ≤0.25 | ≤0.25 | ≤0.25 |
| | gonorrhoeae | | | | |
| 185 | Neisseria | ≤0.25 | ≤0.25 | ≤0.25 | ≤0.25 |
| | gonorrhoeae | | | | |
| 186 | Neisseria | ≤0.25 | ≤0.25 | ≤0.25 | ≤0.25 |
| | gonorrhoeae | | | | |
| 187 | Neisseria | ≤0.25 | ≤0.25 | ≤0.25 | ≤0.25 |
| | gonorrhoeae | | | | |
| 188 | Neisseria | ≤0.25 | ≤0.25 | ≤0.25 | ≤0.25 |
| | gonorrhoeae | | | | |
| 189 | Neisseria | ≤0.25 | ≤0.25 | ≤0.25 | ≤0.25 |
| | gonorrhoeae | | | | |
| 190 | Neisseria | ≤0.25 | ≤0.25 | ≤0.25 | ≤0.25 |
| | gonorrhoeae | | | | |
| 191 | Neisseria | ≤0.25 | ≤0.25 | ≤0.25 | ≤0.25 |
| | gonorrhoeae | | | | |
| 192 | Neisseria | ≤0.25 | ≤0.25 | ≤0.25 | ≤0.25 |
| | gonorrhoeae | | | | |
| 193 | Neisseria | ≤0.25 | ≤0.25 | ≤0.25 | ≤0.25 |
| | gonorrhoeae | | | | |
| 194 | Neisseria | ≤0.25 | ≤0.25 | ≤0.25 | ≤0.25 |
| | gonorrhoeae | | | | |
| 195 | Neisseria | ≤0.25 | ≤0.25 | ≤0.25 | ≤0.25 |
| | gonorrhoeae | | | | |
| 196 | Neisseria | ≤0.25 | ≤0.25 | ≤0.25 | ≤0.25 |
| | gonorrhoeae | | | | |
| 198 | Neisseria | ≤0.25 | ≤0.25 | ≤0.25 | ≤0.25 |
| | gonorrhoeae | | | | |
| 199 | Neisseria | ≤0.25 | ≤0.25 | ≤0.25 | ≤0.25 |
| | gonorrhoeae | | | | |
| 200 | Neisseria | ≤0.25 | ≤0.25 | ≤0.25 | ≤0.25 |
| | gonorrhoeae | | | | |
| 201 | Neisseria | ≤0.25 | ≤0.25 | ≤0.25 | ≤0.25 |
| | gonorrhoeae | | | | |
| 202 | Neisseria | ≤0.25 | ≤0.25 | ≤0.25 | ≤0.25 |
| | gonorrhoeae | | | | |
| 203 | Neisseria | ≤0.25 | ≤0.25 | ≤0.25 | ≤0.25 |
| | gonorrhoeae | | | | |
| 204 | Neisseria | ≤0.25 | ≤0.25 | 0.5 | 0.5 |
| | gonorrhoeae | | | | |
| 205 | Neisseria | ≤0.25 | ≤0.25 | ≤0.25 | ≤0.25 |
| | gonorrhoeae | | | | |
| 206 | Neisseria | ≤0.25 | ≤0.25 | ≤0.25 | ≤0.25 |
| | gonorrhoeae | | | | |
| 207 | Neisseria | ≤0.25 | ≤0.25 | ≤0.25 | ≤0.25 |
| | gonorrhoeae | | | | |
| 208 | Neisseria | ≤0.25 | ≤0.25 | ≤0.25 | ≤0.25 |
| | gonorrhoeae | | | | |
| 209 | Neisseria | ≤0.25 | ≤0.25 | ≤0.25 | ≤0.25 |
| | gonorrhoeae | | | | |
| 210 | Neisseria | ≤0.25 | ≤0.25 | ≤0.25 | ≤0.25 |
| | gonorrhoeae | | | | |
| 211 | Neisseria | ≤0.25 | ≤0.25 | ≤0.25 | ≤0.25 |
| | gonorrhoeae | | | | |
| 212 | Neisseria | ≤0.25 | ≤0.25 | ≤0.25 | ≤0.25 |
| | gonorrhoeae | | | | |
| 213 | Neisseria | ≤0.25 | ≤0.25 | ≤0.25 | ≤0.25 |
| | gonorrhoeae | | | | |
| 214 | Neisseria | ≤0.25 | ≤0.25 | ≤0.25 | ≤0.25 |
| | gonorrhoeae | | | | |

1. A halogenated salicylanilide, or a pharmaceutically acceptable salt or solvate thereof, for use in the treatment, including prevention, of an infection caused by Neisseria gonococcus in a subject, wherein the subject is not treated concurrently with (i) a bacterial efflux pump inhibitor and/or (ii) an agent selected from the group consisting of polymyxins and gramicidins.

2. A halogenated salicylanilide selected from the group consisting of closantel, rafoxanide, oxyclozanide, niclosamide, and pharmaceutically acceptable salts or solvates thereof, for use in the topical treatment, including prevention, of an infection caused by Neisseria gonococcus in a subject.

3. The halogenated salicylanilide, or a pharmaceutically acceptable salt or solvate thereof, for the use of claim 1 or claim 2, wherein the infection by Neisseria gonococcus causes an infectious disease.

4. The halogenated salicylanilide, or a pharmaceutically acceptable salt or solvate thereof, for the use of any of claims 1 to claim 3, wherein the infection by Neisseria gonococcus is (i) a urogenital, an anorectal, a pharyngeal and/or a conjunctival gonococcal infection, or (ii) a disseminated gonococcal infection.

5. The halogenated salicylanilide, or a pharmaceutically acceptable salt or solvate thereof, for the use of claim 4, wherein a disseminated gonococcal infection causes one or more complications in the subject selected from skin lesions, tenosynovitis, perihepatitis, arthralgia, arthritis, arthritis-dermatitis syndrome, meningitis and endocarditis.

6. The halogenated salicylanilide, or a pharmaceutically acceptable salt or solvate thereof, for the use of claim 3, wherein the infectious disease caused by Neisseria gonococcus is selected from adult gonococcal conjunctivitis, pediatric gonococcal conjunctivitis or neonatal gonococcal conjunctivitis, in particular neonatal gonococcal conjunctivitis.

7. The halogenated salicylanilide, or a pharmaceutically acceptable salt or solvate thereof, for the use of claim 3, wherein the infectious disease caused by Neisseria gonococcus is anorectal gonorrhoea.

8. The halogenated salicylanilide, or a pharmaceutically acceptable salt or solvate thereof, for the use of claim 3, wherein the infectious disease caused by Neisseria gonococcus is urogenital gonorrhoea.

9. A halogenated salicylanilide, or a pharmaceutically acceptable salt or solvate thereof, for use in the topical treatment, including prevention, of conjunctival gonococcal infection, in particular neonatal gonococcal conjunctivitis.

10. A halogenated salicylanilide, or a pharmaceutically acceptable salt or solvate thereof, for use in the topical treatment, including prevention, of anorectal gonococcal infection.

11. A halogenated salicylanilide, or a pharmaceutically acceptable salt or solvate thereof, for use in a method of preventing or inhibiting the transmission or spread of a Neisseria gonococcus infection, the method comprising administering an effective amount of the halogenated salicylanilide, or a pharmaceutically acceptable salt or solvate thereof, to a subject with a Neisseria gonococcus infection, wherein the subject is not administered concurrently with (i) a bacterial efflux pump inhibitor and/or (ii) an agent selected from the group consisting of polymyxins and gramicidins.

12. A halogenated salicylanilide, or a pharmaceutically acceptable salt or solvate thereof, for use in a method of preventing or inhibiting the transmission or spread of a conjunctival gonococcal infection, the method comprising topically administering an effective amount of the halogenated salicylanilide, or a pharmaceutically acceptable salt or solvate thereof, to the subject with the conjunctival gonococcal infection.

13. A halogenated salicylanilide, or a pharmaceutically acceptable salt or solvate thereof, for use in a method of preventing or inhibiting the transmission or spread of a gonococcal infection from a mother to an infant, the method comprising topically administering an effective amount of the halogenated salicylanilide, or a pharmaceutically acceptable salt or solvate thereof, to the infant's eye to prevent a conjunctival gonococcal infection

14. A halogenated salicylanilide, or a pharmaceutically acceptable salt or solvate thereof, for use in a method of preventing or inhibiting the transmission or spread of an anorectal gonococcal infection, the method comprising topically administering an effective amount of the halogenated salicylanilide, or a pharmaceutically acceptable salt or solvate thereof, to the subject with the anorectal gonococcal infection.

15. A halogenated salicylanilide, or a pharmaceutically acceptable salt or solvate thereof, for use in a method of preventing or inhibiting recurrence of Neisseria gonococcus infection in a subject with a Neisseria gonococcus infection, the method comprising administering an effective amount of the halogenated salicylanilide, or a pharmaceutically acceptable salt or solvate thereof to the subject, wherein the subject is not administered concurrently with (i) a bacterial efflux pump inhibitor and/or (ii) an agent selected from the group consisting of polymixins and gramicidins.

16. A halogenated salicylanilide, or a pharmaceutically acceptable salt or solvate thereof, for use in a method of preventing or inhibiting recurrence of conjunctival gonococcal infection in a subject, the method comprising topically administering an effective amount of the halogenated salicylanilide, or a pharmaceutically acceptable salt or solvate thereof, to the subject.

17. A halogenated salicylanilide, or a pharmaceutically acceptable salt or solvate thereof, for use in a method of preventing or inhibiting recurrence of anorectal gonococcal infection in a subject, the method comprising topically administering an effective amount of the halogenated salicylanilide, or a pharmaceutically acceptable salt or solvate thereof, to the subject.

18. The halogenated salicylanilide, or a pharmaceutically acceptable salt or solvate thereof, for the use of any of claims 1 to 17, wherein the Neisseria gonococcus is resistant to an antibiotic agent other than the halogenated salicylanilide.

19. The halogenated salicylanilide, or a pharmaceutically acceptable salt or solvate thereof, for the use of claim 18, wherein the Neisseria gonococcus is a Neisseria gonococcus strain that is resistant to one or more antibiotic agents selected from sulfonamides, penicillins, tetracyclines (e.g. doxycycline), quinolones (such as fluoroquinolones, e.g. ciprofloxacin, ofloxacin, and levofloxacin), cephalosporins (e.g. cephalexin, cefixime and ceftriaxone) or macrolides (e.g. azithromycin).

20. The halogenated salicylanilide, or a pharmaceutically acceptable salt or solvate thereof, for the use of claim 18, wherein the Neisseria gonococcus is a Neisseria gonococcus strain that is resistant to ceftriaxone and/or azithromycin.

21. The halogenated salicylanilide, or a pharmaceutically acceptable salt or solvate thereof, for the use of any of claims 1 to 20, wherein the subject has a recurrent Neisseria gonococcus infection.

22. The halogenated salicylanilide, or a pharmaceutically acceptable salt or solvate thereof, for the use of claim 21, wherein the Neisseria gonococcus infection has recurred after prior treatment of the subject with an antibiotic selected from sulfonamides, penicillins, tetracyclines (e.g. doxycycline), quinolones (such as fluoroquinolones, e.g. ciprofloxacin, ofloxacin, and levofloxacin), cephalosporins (e.g. cephalexin, cefixime and ceftriaxone) or macrolides (e.g. azithromycin).

23. The halogenated salicylanilide, or a pharmaceutically acceptable salt or solvate thereof, for the use of any of claims 1 to 20, wherein the Neisseria gonococcus infection has not been treated with an antibiotic prior to administration of the halogenated salicylanilide to the subject.

24. The halogenated salicylanilide, or a pharmaceutically acceptable salt or solvate thereof, for the use of any preceding claim, wherein the halogenated salicylanilide is topically administered to the subject.

25. The halogenated salicylanilide, or a pharmaceutically acceptable salt or solvate thereof, for the use of claim 24, wherein the halogenated salicylanilide is topically administered to the subject using a topical formulation selected from a solution, an emulsion, a suspension, a cream, a foam, a gel, a lotion, an ointment or a suppository, said topical formulation comprising the halogenated salicylanilide.

26. The halogenated salicylanilide, or a pharmaceutically acceptable salt or solvate thereof, for the use of claim 24, wherein the halogenated salicylanilide is topically administered to the subject using a device, such as a syringe, a condom or a vaginal pessary, coated or filled with the halogenated salicylanilide or a composition comprising the halogenated salicylanilide.

27. The halogenated salicylanilide, or a pharmaceutically acceptable salt or solvate thereof, for the use of any preceding claim, wherein the subject is a human or warm blooded animal, optionally wherein the subject is a human.

28. The halogenated salicylanilide for the use of any of claims 1 or 3 to 27, wherein the halogenated salicylanilide is of the formula (I): [chemical structure]
wherein
X is O or S;
R¹ and R² are at each occurrence independently selected from halo;
R³ and R⁴ are at each occurrence independently selected from H, C₁₋₆ alkyl, —OR^{A1}, —NO₂ and —CN;
R⁵ is H or -L¹-R⁷;
R⁶ is H or —C(O)R^{A2};
L¹ is selected from a bond, O, S, or —(CR^{A3}R^{B})ₒ—, wherein o is 1 or 2;
R⁷ is phenyl, unsubstituted or substituted with 1, 2, or 3 groups selected from halo, C₁₋₄ alkyl, —OR^{A4}, —NO₂ and —CN;
R^{A1}, R^{A2}, R^{A3} and R^{A4} are at each occurrence independently selected from H and C₁₋₄ alkyl;
R^{B} is at each occurrence selected from H, C₁₋₄ alkyl and —CN;
n and p are each independently selected from 0, 1, 2, 3 or 4, with the proviso that n+p is at least 1;
t and v are independently selected from 0, 1 and 2;
or a pharmaceutically acceptable salt or solvate thereof.

29. The halogenated salicylanilide for the use of claim 28, wherein the halogenated salicylanilide is of the formula (II): [chemical structure]
or a pharmaceutically acceptable salt or solvate thereof.

30. The halogenated salicylanilide, or a pharmaceutically acceptable salt or solvate thereof, for the use of claim 28 or claim 29, wherein X is O.

31. The halogenated salicylanilide, or a pharmaceutically acceptable salt or solvate thereof, for the use of any of claims 28 to 30, wherein R⁶ is H.

32. The halogenated salicylanilide, or a pharmaceutically acceptable salt or solvate thereof, for the use of any of claims 28 to 31, wherein R³ and R⁴ are at each occurrence independently selected from H, C₁₋₄ alkyl, —OR^{A1} and —NO₂.

33. The halogenated salicylanilide, or a pharmaceutically acceptable salt or solvate thereof, for the use of any of claims 28 to 32, wherein L¹ is selected from O, —CH₂— and —CH(CN)—.

34. The halogenated salicylanilide, or a pharmaceutically acceptable salt or solvate thereof, for the use of any of claims 28 to 33, wherein R⁷ is phenyl unsubstituted or substituted with 1, 2 or 3 groups selected from halo.

35. The halogenated salicylanilide for the use of any of claims 1 or 3 to 27, wherein the halogenated salicylanilide is selected from: [chemical structure] [chemical structure]
or a pharmaceutically acceptable salt or solvate thereof.

36. The halogenated salicylanilide for the use of any of claims 1 or 3 to 27, wherein the halogenated salicylanilide is selected from niclosamide, clioxanide, closantel, oxyclozanide, rafoxanide, tribromosalan, or a pharmaceutically acceptable salt or solvate thereof.

37. The halogenated salicylanilide for the use of any of claims 1 to or 3 to 27, wherein the halogenated salicylanilide is selected from niclosamide, closantel, oxyclozanide, rafoxanide, or a pharmaceutically acceptable salt or solvate thereof.

38. The halogenated salicylanilide for the use of any of claims 1 to 27, wherein the halogenated salicylanilide is niclosamide or a pharmaceutically acceptable salt thereof.

39. The halogenated salicylanilide for the use of any of claims 1 to 27, wherein the halogenated salicylanilide is oxyclozanide or a pharmaceutically acceptable salt thereof.

40. A method of treating, including preventing, an infection caused by Neisseria gonococcus bacteria in a subject, the method comprising administering to said subject an effective amount of a halogenated salicylanilide, or a pharmaceutically acceptable salt or solvate thereof, wherein the subject is not administered concurrently with (i) a bacterial efflux pump inhibitor and/or (ii) an agent selected from the group consisting of polymyxins and gramicidins.

41. The method of claim 40, wherein the infection caused by Neisseria gonococcus causes an infectious disease.

42. The method of claim 40 or claim 41, wherein the infection caused by Neisseria gonococcus is (i) a urogenital, an anorectal, a pharyngeal and/or a conjunctival gonococcal infection or (ii) a disseminated gonococcal infection.

43. The method of claim 42, wherein a disseminated gonococcal infection is causing one or more complications selected from skin lesions, tenosynovitis, perihepatitis, arthralgia, arthritis, arthritis-dermatitis syndrome, meningitis, or endocarditis.

44. The method of claim 41, wherein the infectious disease caused by Neisseria gonococcus is selected from adult gonococcal conjunctivitis or neonatal gonococcal conjunctivitis, in particular neonatal gonococcal conjunctivitis.

45. The method of claim 41, wherein the infectious disease caused by Neisseria gonococcus is urogenital gonorrhoea.

46. The method of claim 41, wherein the infectious disease caused by Neisseria gonococcus is anorectal gonorrhoea.

47. A method of treating of a conjunctival gonococcal infection, in particular neonatal gonococcal conjunctivitis, in a subject, the method comprising topically administering to said subject an effective amount of a halogenated salicylanilide, or a pharmaceutically acceptable salt or solvate thereof.

48. A method of treating of an anorectal gonococcal infection in a subject, the method comprising topically administering to said subject an effective amount of a halogenated salicylanilide, or a pharmaceutically acceptable salt or solvate thereof.

49. A method for preventing or inhibiting the transmission or spread of a Neisseria gonococcus infection, the method comprising administering an effective amount of a halogenated salicylanilide, or a pharmaceutically acceptable salt or solvate thereof, to a subject with a Neisseria gonococcus infection, wherein the subject is not administered concurrently with (i) a bacterial efflux pump inhibitor and/or (ii) an agent selected from the group consisting of polymyxins and gramicidins.

50. A method for preventing or inhibiting the transmission or spread of a Neisseria gonococcus infection from a mother to an infant, the method comprising topically administering an effective amount of a halogenated salicylanilide, or a pharmaceutically acceptable salt or solvate thereof, to the infant's eye to prevent a conjunctival gonococcal infection.

51. A method for preventing or inhibiting the transmission or spread of a conjunctival gonococcal infection, the method comprising topically administering an effective amount of a halogenated salicylanilide, or a pharmaceutically acceptable salt or solvate thereof, to a subject with a conjunctival gonococcal infection.

52. A method for preventing or inhibiting the transmission or spread of an anorectal gonococcal infection, the method comprising topically administering an effective amount of a halogenated salicylanilide, or a pharmaceutically acceptable salt or solvate thereof, to a subject with an anorectal gonococcal infection.

53. The method of any of claims 40 to 52, wherein the subject has a recurrent Neisseria gonococcus infection.

54. The method of claim 53, wherein the Neisseria gonococcus infection has recurred after being treated with an antibiotic selected sulfonamides, penicillins, tetracyclines (e.g. doxycycline), quinolones (such as fluoroquinolones, e.g. ciprofloxacin, ofloxacin, and levofloxacin), cephalosporins (e.g. cephalexin, cefixime and ceftriaxone) or macrolides (e.g. azithromycin).

55. A method of preventing or inhibiting recurrence of Neisseria gonococcus infection in a subject with a Neisseria gonococcus infection, the method comprising administering to said subject an effective amount of a halogenated salicylanilide, or a pharmaceutically acceptable salt or solvate thereof, wherein the subject is not administered concurrently with (i) a bacterial efflux pump inhibitor and/or (ii) an agent selected from the group consisting of polymyxins and gramicidins.

56. A method of preventing or inhibiting recurrence of conjunctival gonococcal infection in a subject with a conjunctival gonococcal infection, the method comprising topically administering to said subject an effective amount of a halogenated salicylanilide, or a pharmaceutically acceptable salt or solvate thereof.

57. A method of preventing or inhibiting recurrence of anorectal gonococcal infection in a subject with an anorectal gonococcal infection, the method comprising topically administering to said subject an effective amount of a halogenated salicylanilide, or a pharmaceutically acceptable salt or solvate thereof.

58. The method of any one claims 40 to 57, wherein the Neisseria gonococcus is resistant to an antibiotic agent other than the halogenated salicylanilide.

59. The method of claim 58, wherein the Neisseria gonococcus is resistant to an antibiotic agent selected from sulfonamides, penicillins, tetracyclines (e.g. doxycycline), quinolones (such as fluoroquinolones, e.g. ciprofloxacin, ofloxacin, and levofloxacin), cephalosporins (e.g. cephalexin, cefixime and ceftriaxone) or macrolides (e.g. azithromycin).

60. The method of claim 58, wherein the Neisseria gonococcus is a Neisseria gonococcus strain that is resistant to ceftriaxone and/or azithromycin.

61. The method of any of claims 40 to 60, wherein the Neisseria gonococcus infection has not been treated with an antibiotic prior to administration of the halogenated salicylanilide to the subject.

62. The method of any of claims 40 to 61, wherein the halogenated salicylanilide is topically administered to the subject.

63. The method of claim 62, wherein the halogenated salicylanilide is topically administered to the subject using a topical formulation selected from a solution, an emulsion, a suspension, a cream, a foam, a gel, a lotion, an ointment or a suppository, said topical formulation comprising the halogenated salicylanilide.

64. The method of claim 62, wherein the halogenated salicylanilide is topically administered to the subject using a device, such as a syringe, a condom or a vaginal pessary, coated or filled with the halogenated salicylanilide.

65. The method of any of claims 40 to 64, wherein the halogenated salicylanilide is administered to the subject concurrently with another therapeutic agent provided said other therapeutic agent is not a bacterial efflux pump inhibitor or an agent selected from the group consisting of polymyxins and gramicidins.

66. The method of any of claims 40 to 65, wherein the halogenated salicylanilide is administered to the subject concurrently with another therapeutic agent which is an antibiotic active against Chlamydia trachomatis.

67. The method of any of claims 40 to 66, wherein the subject is a human or warm blooded animal, optionally wherein the subject is a human.

68. The method of any of claims 40 to 67, wherein the halogenated salicylanilide is as defined in any of claims 28 to 39.

69. Use of a halogenated salicylanilide, or a pharmaceutically acceptable salt or solvate thereof, for the manufacture of a medicament for the treatment of an infection in a subject caused by Neisseria gonococcus bacteria, said medicament lacking a bacterial efflux pump inhibitor and lacking an agent selected from the group consisting of polymyxins and gramicidins.

70. Use of a halogenated salicylanilide, or a pharmaceutically acceptable salt or solvate thereof, for the manufacture of a medicament for the topical treatment of conjunctival gonococcal infection, in particular neonatal gonococcal conjunctivitis, in a subject.

71. Use of a halogenated salicylanilide, or a pharmaceutically acceptable salt or solvate thereof, for the manufacture of a medicament for the topical treatment of anorectal gonococcal infection in a subject.

72. Use of any of claims 69 to 71, wherein the halogenated salicylanilide is as defined in any of claims 28 to 39.

73. A condom coated with a halogenated salicylanilide, or a pharmaceutically acceptable salt or solvate thereof, wherein the halogenated salicylanilide is as defined in any of claims 28 to 39.

74. A vaginal pessary coated with a halogenated salicylanilide, or a pharmaceutically acceptable salt or solvate thereof, wherein the halogenated salicylanilide is as defined in any of claims 28 to 39.

75. A disposable syringe pre-filled with a topical formulation comprising a halogenated salicylanilide, or a pharmaceutically acceptable salt or solvate thereof, wherein the halogenated salicylanilide is as defined in any of claims 28 to 39.

76. The disposable syringe of claim 75, wherein the topical formulation is selected from a solution, an emulsion, a suspension, a cream, a foam, a gel, a lotion or an ointment.
